(19) United States
(12) Patent Application Publication
(10) Pub. No.: US 2016/0046984 A1
(43) Pub. Date: Feb. 18, 2016

(54) Robust Detection of Nucleic Acids in Situ

(71) Applicant: Affymetrix, Inc., Santa Clara, CA (US)

(72) Inventors: Quan N. Nguyen, San Ramon, CA (US); Yunqing Ma, San Jose, CA (US); Botoul Maqsodi, Fremont, CA (US); Manoj Gandhi, Dublin, CA (US)

(21) Appl. No.: 14/826,907

(22) Filed: Aug. 14, 2015

Related U.S. Application Data
(60) Provisional application No. 62/037,983, filed on Aug. 15, 2014.

(51) Int. Cl. C12Q 1/68

(52) U.S. Cl. CPC C12Q 1/6841

(57) ABSTRACT

Methods of detecting nucleic acids, including methods of detecting nucleic acids in situ, are provided. The methods can detect even target nucleic acids that are partially degraded and/or masked by extensive crosslinking. Compositions, kits, and systems related to the methods are also described

### CROSS-REFERENCE TO RELATED APPLICATIONS

This application claims priority to and benefit of a prior U.S. Provisional Application No. 62/037,983, by Nguyen, et al., filed Aug. 15, 2014. The full disclosure of the prior application is incorporated herein by reference.

### FIELD OF THE INVENTION

The present invention is in the field of nucleic acid analysis. More particularly, the invention relates to methods for detection of nucleic acid analytes in situ or after capture from solution onto a solid support. The invention also includes compositions and kits related to the methods.

### BACKGROUND OF THE INVENTION

In situ hybridization (ISH) uses a labeled probe to detect and typically localize a particular nucleic acid sequence in a tissue section, in the entirety of a small tissue (e.g., a whole mount of a Drosophila embryo or plant seed), or in cells (e.g., circulating tumor cells). Although in situ hybridization is a powerful and widely used tool for detecting and localizing specific nucleic acids in cells or tissues, improved techniques for detection of low copy number targets, detection of targets whose expression levels vary widely, detection of RNAs unintentionally degraded during sample preparation, detection from aged, poorly preserved, or poorly or inconsistently prepared samples, and detection using automated platforms are highly desirable.

Kenney (U.S. Pat. No. 7,033,758) has noted in Highly Sensitive Gene Detection and Localization Using In Situ Branched-DNA Hybridization, that such detections are limited to, e.g., samples wherein not less than 400 nucleic acid bases are available in the target region. However, the described techniques were not adequate in situ for very short nucleic acids, degraded nucleic acids, or nucleic acid targets masked by binding proteins.

The present invention meets these and other needs by providing, inter alia, methods and compositions for robust detection of nucleic acids in situ. A complete understanding of the invention will be obtained upon review of the following.

### SUMMARY OF THE INVENTION

In one aspect, the present invention provides methods of detecting a target nucleic acid. Novel label extender configurations are employed in some embodiments. The methods facilitate robust detection of nucleic acids, particularly in situ and/or under difficult conditions. Compositions and kits related to the methods are also provided.

A first general class of embodiments provides methods of detecting a target nucleic acid. In the methods, a sample comprising the target nucleic acid is provided. One or more label extenders are hybridized to the target nucleic acid. One or more copies of a preamplifier are hybridized to each of the label extenders, wherein each copy of the preamplifier hybridizes to a single one of the label extenders (i.e. without hybridizing to another label extender). Multiple copies of a label are bound to each copy of the preamplifier. The hybridizing and binding steps, which can be performed simultaneously or sequentially in any convenient order as desired, capture multiple copies of the label to the target nucleic acid. A signal produced by the label is detected. Since the label is captured to the target nucleic acid, detection of the label detects the target nucleic acid. Optionally, localization and/or quantitation are performed (e.g., to determine the amount of the target nucleic acid present in a particular tissue, cell type, organelle, subcellular location, or the like).

In one class of embodiments, two or more copies of the preamplifier are hybridized to each of the label extenders. For example, three or more, four or more, or even five or more copies of the preamplifier can be hybridized to each of the label extenders.

Each label extender typically includes a polynucleotide sequence L-1 that is complementary to a polynucleotide sequence in the target nucleic acid. In one class of embodiments, L-1 is less than 15 nucleotides in length. For example, L-1 can be between five and 13 nucleotides in length (inclusive), e.g., between seven and ten nucleotides in length (inclusive). In embodiments in which two or more label extenders are employed, the label extenders can hybridize to contiguous or noncontiguous regions of the target nucleic acid.

The methods can be used to detect the presence of the target nucleic acid in essentially any type of sample. Similarly, the target nucleic acid can be essentially any desired nucleic acid, for example, a DNA or an RNA (e.g., an mRNA or microRNA). The target nucleic acid can be partially degraded or too short to be detected using other, conventional techniques. For example, the target nucleic acid (or optionally the target region of the target nucleic acid within which all of the label extenders hybridize, in embodiments in which the entire target nucleic acid is longer) can be 400 nucleotides or less in length, e.g., 300 nucleotides or less, 200 nucleotides or less, 100 nucleotides or less, 50 nucleotides or less, or even 25 nucleotides or less in length.

In a particularly preferred aspect, the methods permit detection of nucleic acids in situ. Thus, in one class of embodiments, the sample comprises a cell comprising the target nucleic acid, and the methods include hybridizing the one or more label extenders to the target nucleic acid in the cell. Other hybridization and binding steps are also performed in the cell, as is detection. The sample is optionally a small whole tissue or a tissue section comprising the cell, e.g., a formalin-fixed, paraffin-embedded (FFPE) tissue section.

The methods can permit pretreatment of the sample to unmask the target nucleic acid to be performed at lower temperature and/or lower protease concentration, thereby better preserving cell and tissue morphology. For example, the sample can be maintained at a temperature of 85° C. or less prior to the hybridizing, binding, and detecting steps, e.g., between 50° C. and 85° C., e.g., for 3 to 120 minutes. FFPE samples such as FFPE tissue sections are typically dewaxed, rehydrated, and optionally briefly dried prior to uncrosslinking and permeabilization. The methods permit treatment steps following the dewaxing, rehydrating, and optional drying steps to be performed at lower temperature. Thus, in one class of embodiments, the sample is an FFPE tissue section that is dewaxed and rehydrated prior to the hybridizing, binding, and detecting steps, and after the dewaxing and rehydrating step and prior to the hybridizing, binding, and detecting steps, the sample is maintained at a temperature of 37° C. or less. For example, the sample can be maintained at room temperature (e.g., 23° C. or 25° C.).

Permeabilization is optionally achieved without the addition of any exogenous protease. In such embodiments, permeabilization can be achieved by heating, e.g., at low temperature as noted above, and/or by addition of a permeabilizing agent. In one exemplary class of embodiments, the sample is incubated in a solution comprising a detergent or amphipathic glycoside at 0.01%-0.2% (v/v) prior to the hybridizing, binding, and detecting steps. Suitable detergents and amphipathic glycosides are known in the art, and include, but are not limited to, saponin, Triton™ X-100, digitonin, Leucoperm™, and Tween® 20. The solution optionally also comprises other solvents and reagents, e.g., acetone, methanol, and/or formamide.

In other embodiments, protease treatment that is gentler than that generally required with other techniques for in situ detection can be employed. For example, the sample can be incubated with proteinase K at a concentration of less than 1 μg/ml (e.g., 0.2-1 μg/ml or 20-100 ng/ml) prior to the hybridizing, binding, and detecting steps. Other suitable proteases are known in the art and can be employed in the methods, e.g., trypsin, pepsin, and protease type XIV. In one aspect, the samples are exposed only to a gentle pretreatment the cells or tissues are exposed to less than 1 μg/ml of protease at a temperature of 85° C. or less for 10 minutes or less. Optionally, the gentle pretreatment does not include exposure of the cells or tissues to an organic solvent (e.g., anhydrous) or exposure to an aldehyde (e.g., pretreatment without a cross-linking step).

At any of various steps, materials not captured to the target nucleic acid are optionally separated from the target nucleic acid. Thus, the methods can include washing the cell, with or without agitation, to remove materials that are not hybridized or bound to the target nucleic acid. As just one example, the methods can include hybridizing the one or more copies of the preamplifier to each of the label extenders, then washing the cell, e.g., without agitation, to remove any copies of the preamplifier that are not hybridized to the label extenders; hybridizing copies of an intermediate amplifier to each copy of the preamplifier, then washing the cell to remove any copies of the intermediate amplifier that are not hybridized to the preamplifiers; hybridizing copies of an amplification multimer to each copy of the intermediate amplifier, then washing the cell to remove any copies of the amplification multimer that are not hybridized to the intermediate amplifiers; and hybridizing copies of a label probe to each copy of the amplification multimer, then washing the cell to remove any copies of the label probe that are not hybridized to the amplification multimers.

Binding multiple copies of the label to each copy of the preamplifier involves various steps, depending on the configuration of the label probe system. For an exemplary class of embodiments in which the label probe system includes the preamplifier, an intermediate amplifier, an amplification multimer, and a label probe, the methods include hybridizing multiple copies of the intermediate amplifier to each copy of the preamplifier, hybridizing multiple copies of the amplification multimer to each copy of the intermediate amplifier, and hybridizing multiple copies of the label probe to each copy of the amplification multimer. In one class of embodiments, each copy of the label probe comprises a copy of the label (e.g., one or more labels). In another class of embodiments, the label probe is configured to bind to the label, and the methods include binding a copy of the label to each copy of the label probe. In other exemplary classes of embodiments, the label probe system includes the preamplifier, an amplification multimer, and a label probe, or the preamplifier, a first intermediate amplifier, a second intermediate amplifier that bridges the first intermediate amplifier and an amplification multimer, the amplification multimer, and a label probe.

Suitable labels and techniques for detection thereof are known in the art. In one class of embodiments, the label is an enzyme. In this class of embodiments, detecting a signal produced by the label optionally comprises providing a chromogenic substrate for the enzyme and detecting a colored product produced by action of the enzyme on the substrate.

Another general class of embodiments provides methods of detecting a target nucleic acid in situ. In the methods, a sample comprising a cell comprising the target nucleic acid is provided. One or more label extenders are hybridized to the target nucleic acid in the cell. Two or more copies of a preamplifier are hybridized to each of the label extenders, wherein each copy of the preamplifier hybridizes to a single one of the label extenders. Multiple copies of a label are bound to each copy of the preamplifier. A signal produced by the label is detected, and the target nucleic acid is thereby detected.

Essentially all of the features noted for the embodiments above apply to these embodiments as well, as relevant; for example, with respect to configuration of the label extenders, configuration of the label probe system, type of label, washing, type of sample, type and size of target nucleic acid, and/or the like.

Yet another general class of embodiments provides methods of detecting a target nucleic acid in situ. In the methods, a sample comprising a cell comprising the target nucleic acid is provided. One or more label extenders are hybridized to the target nucleic acid in the cell. Each of the label extenders comprises a polynucleotide sequence L-1 that is complementary to a polynucleotide sequence in the target nucleic acid. L-1 is less than 15 nucleotides in length, e.g., between five and 13 nucleotides in length (inclusive), e.g., between seven and ten nucleotides in length (inclusive). One or more copies of a preamplifier are hybridized to each of the label extenders, wherein each copy of the preamplifier hybridizes to a single one of the label extenders. Multiple copies of a label are bound to each copy of the preamplifier. A signal produced by the label is detected, and the target nucleic acid is thereby detected.

Essentially all of the features noted for the embodiments above apply to these embodiments as well, as relevant; for example, with respect to configuration of the label extenders, configuration of the label probe system, type of label, washing, type of sample, type and size of target nucleic acid, and/or the like. Thus, in one class of embodiments, two or more copies of the preamplifier are hybridized to each of the label extenders. In one class of embodiments, the label is an enzyme, and detecting a signal produced by the label comprises providing a chromogenic substrate for the enzyme and detecting a colored product produced by action of the enzyme on the substrate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically illustrates a typical standard sandwich bDNA assay.

FIG. 2 schematically illustrates sandwich bDNA assays in which two label extenders bind to each preamplifier. Panel A schematically depicts a ZZ label extender configuration. Panel B schematically depicts a cruciform label extender configuration.

FIG. 3 Panels A and B compare efficacy of label extender binding to a target nucleic acid that is partially blocked by proteins and partially degraded for two different label extender/preamplifier configurations. Panel A schematically illustrates a configuration in which binding of two label extenders to a preamplifier is required to capture the preamplifier to the target nucleic acid. Panel B schematically illustrates a configuration in which binding of a single label extender to a preamplifier captures the preamplifier to the target nucleic acid.

FIG. 4 Panels A-E illustrate the differences in maximum signal achievable for five different label extender/preamplifier configurations.

FIG. 5 Panels A-D illustrate detection of a 1000 base (Panels A and B) and a 500 base (Panels C and D) region of rat Synpo in situ with two different label extender configurations, ZZ (Panels A and C) and Z1 (Panels B and D). The maximum number of preamplifiers expected to bind in each different configuration is indicated as “# bDNA.”

FIG. 6 Panels A-D illustrate detection of a 50 base (Panels A and B) and a 25 base (Panels C and D) region of rat Synpo in situ with two different label extender configurations, ZZ (Panels A and C) and Z1 (Panels B and D). The maximum number of preamplifiers expected to bind in each different configuration is indicated as “# bDNA.”

FIG. 7 Panels A and B illustrate detection of rat Let7a microRNA in situ with two different label extender configurations, ZZ (Panel A) and Z2 (Panel B). The maximum number of preamplifiers expected to bind in each different configuration is indicated as “# bDNA.”

FIG. 8 Panels A-C illustrate detection of albumin in situ in human liver with three different label extender configurations, ZZ (Panel A), SZ (Panel B), and Z1 (Panel C).

FIG. 9 Panels A and B illustrate detection of albumin in situ with two different label extender configurations, SZ (Panel A) and Z1 (Panel B).

FIG. 10 schematically illustrates one possible configuration of the label probe system. In this example, the label probe system includes a preamplifier, amplification multimer, and label probe. Each label extender includes sequences L-1 (complementary to a sequence of the target RNA) and L-2 (complementary to sequence P-1 of the preamplifier; in this exemplary configuration). Sequence A-1 of the amplification multimer is complementary to sequence P-2 of the preamplifier, and sequence A-2 of the amplification multimer is complementary to sequence LP-1 of the label probe.

FIG. 11 is a series of photos showing effects of various protease concentrations on in situ detection using a single label extender (Z1) bDNA format versus a double label extender (ZZ) bDNA assay format in a ER1 epitope retrieval buffer pH 6. With gentler protease digestion (1:500, 1:1000, 1:2000 and no protease, with the dilutions from a 17 mg/ml stock solution), the liver tissue shows significantly better morphology with retention of the trabecular pattern and no nuclear clearing (arrowheads). However, despite gentler pretreatment, the Albumin Z1 probe set shows a robust staining pattern within the hepatocytes (upper panels) across the different conditions. In contrast, the Albumin ZZ probe set shows significantly reduced and heterogeneous staining (lower panels) across the different conditions. The photomicrographs are based on human liver processed using ER1 epitope retrieval buffer and ViewRNA eZ-L Detection kit on the Leica Bond III automated ISH staining instrument. Protease digestion time was 30 min for all dilutions. Images taken at 20× objective magnification.

FIG. 12 is a series of photos showing effects of various protease concentrations on in situ detection using a single label extender (Z1) bDNA format versus a double label extender (ZZ) bDNA assay format in an ER2 epitope retrieval buffer pH 9. With gentler protease digestion (1:500, 1:1000, 1:2000 and no protease, with the dilutions from a 17 mg/ml stock solution), the liver tissue shows significantly better morphology with retention of the trabecular pattern and no nuclear clearing (arrowheads). However, despite gentler pretreatment, the Albumin Z1 probe set shows a robust staining pattern within the hepatocytes (upper panels) across the different conditions. In contrast, the Albumin ZZ probe set shows significantly reduced and heterogeneous staining (lower panel) across the different conditions. The photomicrographs are of human liver processed using ER2 epitope retrieval buffer and ViewRNA eZ-L Detection kit on the Leica Bond III automated ISH staining instrument. Protease digestion time was 30 min for all dilutions. Images taken at 20× objective magnification.

FIG. 13 presents a pair of photomicrographs showing detection of small nucleic acids (miRNA) using techniques described herein. The two examples show epidermal layers of the skin with presence of miRNA Let7a using the single Z probe design. Human skin shows robust signal associated with the moderate expression of Let7a with 6-20 dots/cell. Target size for Let7a is 22 bases and the miRNA ISH probe consists of a single label extender probe of 22 bases (Z1 design) allowing only one bDNA structure (of a preamplifier, amplifiers and label probes) for signal amplification. Here human skin is processed with 1:150 dilution of protease (from a stock concentration of 2.7 mg/ml) using the ViewRNA Manual Detection kit. Images taken at 40× objective magnification.

FIG. 14 is a schematic diagram of a label probe system including a preamplifier, intermediate amplifiers, amplification multimers (amplifiers), and multiple copies of a label probe.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a molecule” includes a plurality of such molecules, and the like.

The term “about” as used herein indicates the value of a given quantity varies by +/−10% of the value, or optionally +/−5% of the value, or in some embodiments, by +/−1% of the value so described.

The term “nucleic acid” (and the equivalent term “polynucleotide”) encompasses any physical string of monomer units that correspond to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural (e.g., locked nucleic acid, isoG, or isoC nucleotides), and can be unsubstituted, unmodified, substituted or modified. The nucleotides can be linked by phosphodiester bonds, or by phosphorothioate linkages, methylphosphonate linkages, boranophosphate linkages, or the like. Polynucleotides can additionally comprise non-nucleotide elements such as labels, quenchers, blocking groups, or the like. Polynucleotides can be, e.g., single-stranded, partially double-stranded, or completely double-stranded. “Masked” nucleic acids are in association with a protein (a ribosome protein, enhancer protein, histone, etc.) at a region of the nucleic acid so that complementary binding of a nucleic acid probe is blocked at that region. “Degraded” nucleic acids are fragmented from their native state, e.g., due to exposure to light, chemicals, and/or enzymes that break the nucleic acid backbone. Degraded nucleic acids typically have breakages at random sites.

A “target nucleic acid” is a nucleic acid in a sample of interest having one or more sequence portions that are complementary to an assay probe (e.g., label extender or capture extender) nucleic acid, as is well known in the art. A “target region” of a target nucleic acid is a contiguous segment of the target nucleic acid within which one or more of the probes (e.g., label extenders) in a hybridization assay can specifically hybridize. A particular target nucleic acid may have one or more target regions. The number of regions may depend on the design of the corresponding complementary probe(s) (e.g., label extenders) and the portion(s) of the target nucleic acid that may provide a unique sequence (in the context of the particular assay) to interrogate with the probe(s) at issue. For example, where a target nucleic acid is 2000 bases long, but a bDNA assay system includes three different label extenders that hybridize to three target sequences along the target nucleic acid between bases 900 and 1200, the target region for that assay would have a length of 300 bases. The target region may be the same as the length of the target nucleic acid, such as when the target nucleic acid is a microRNA (commonly 22 bases in length) or other relatively short target nucleic acids. bDNA label probe systems typically contact the target DNA (e.g., at a target region) through a complementary L-1 sequence of a label extender.

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York), as well as in Ausubel, infra.

The “Tₘ” (melting temperature) of a nucleic acid duplex under specified conditions (e.g., relevant assay conditions) is the temperature at which half of the base pairs in a population of the duplex are disassociated and half are associated. The Tₘ for a particular duplex can be calculated and/or measured, e.g., by obtaining a thermal denaturation curve for the duplex (where the Tₘ is the temperature corresponding to the midpoint in the observed transition from double-stranded to single-stranded form).

The term “complementary” refers to a polynucleotide (or portion thereof) that forms a stable duplex with its “complement” (a polynucleotide or portion thereof), e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A first polynucleotide that is “capable of hybridizing” (or, equivalently, “configured to hybridize”) to a second polynucleotide comprises a first polynucleotide sequence that is complementary to a second polynucleotide sequence in the second polynucleotide.

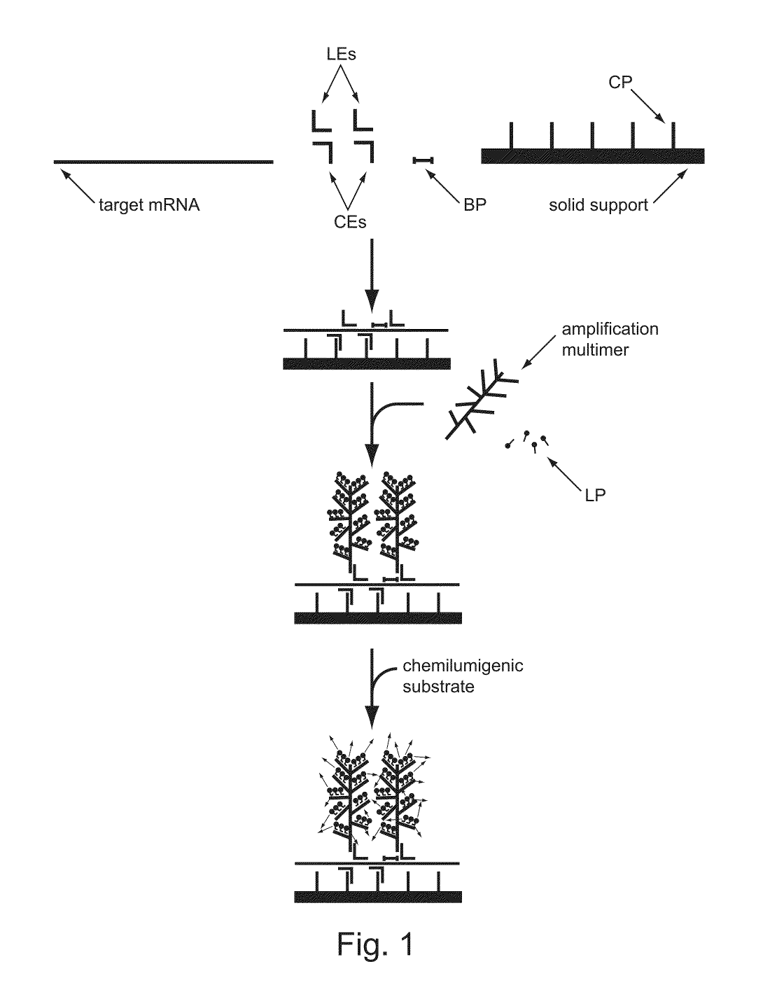

A “capture extender” or “CE” is a polynucleotide that is capable of hybridizing to a target nucleic acid and that is preferably also capable of hybridizing to a capture probe. The capture extender typically has a first polynucleotide sequence C-1, which is complementary to the capture probe, and a second polynucleotide sequence C-3, which is complementary to a polynucleotide sequence of the target nucleic acid. Sequences C-1 and C-3 are typically not complementary to each other. For example, see FIG. 1. The capture extender is preferably single-stranded. Capture extenders are typically associated with a capture probe of an in vitro capture system.

A “capture probe” or “CP” is a polynucleotide that is capable of hybridizing to at least one capture extender and that is tightly bound (e.g., covalently or noncovalently, directly or through a linker, e.g., streptavidin-biotin or the like) to a solid support, a spatially addressable solid support, a slide, a particle, a microsphere, or the like. The capture probe typically comprises at least one polynucleotide sequence C-2 that is complementary to polynucleotide sequence C-1 of at least one capture extender. The capture probe is preferably single-stranded.

A “label extender” or “LE” is a polynucleotide that is capable of hybridizing to a target nucleic acid and to at least one portion of a label probe system. The label extender typically has a first polynucleotide sequence L-1, which is complementary to a polynucleotide sequence of the target nucleic acid, and a second polynucleotide sequence L-2, which is complementary to a polynucleotide sequence of the label probe system (e.g., L-2 can be complementary to a polynucleotide sequence (P−1) of a preamplifier, an amplification multimer (A−1), a label probe, or the like). The label extender is preferably a single-stranded polynucleotide. The label extender optionally includes a linker sequence between L-1 and L-2. In embodiments in which the label extender includes two or more L-2 sequences (e.g., for hybridization of two or more preamplifiers to the label extender), the label extender optionally includes a linker sequence between any neighboring L-2 sequences. Suitable linkers include, but are not limited to, oligo dT stretches, e.g., 5 Ts.

A “label” is a moiety that facilitates detection of a molecule. Common labels in the context of the present invention include fluorescent, luminescent, light-scattering, chromogenic, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Labels include the use of enzymes such as alkaline phosphatase that are conjugated to a polynucleotide probe for use with an appropriate enzymatic substrate, such as fast red or fast blue, which is described within U.S. Pat. Nos. 5,780,227 and 7,033,758. Other enzymatic labels are also contemplated, such as conjugation of horseradish peroxidase to polynucleotide probes for use with 3,3′-Diaminobenzidine (DAB). Many labels are commercially available and can be used in the context of the invention.

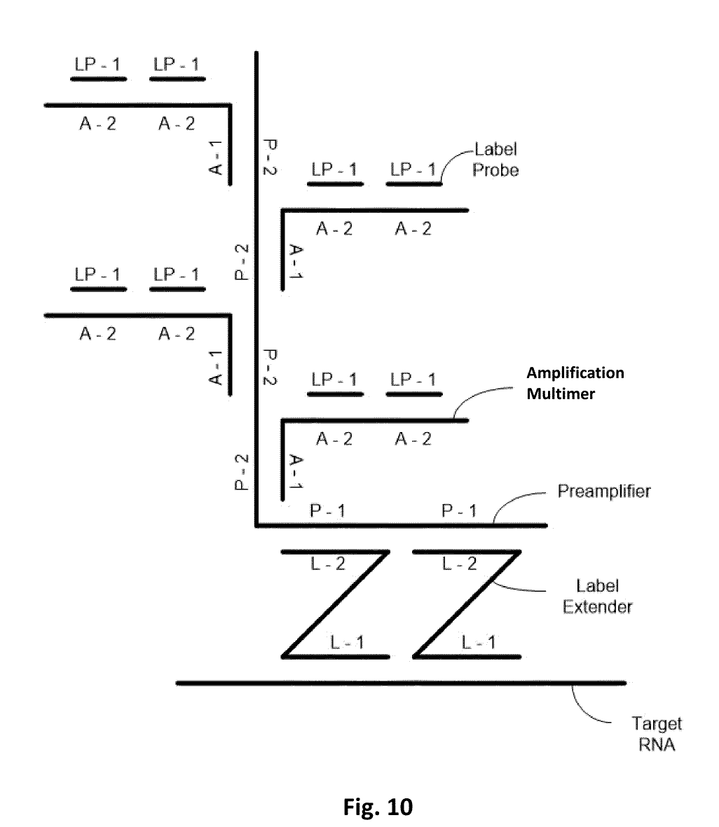

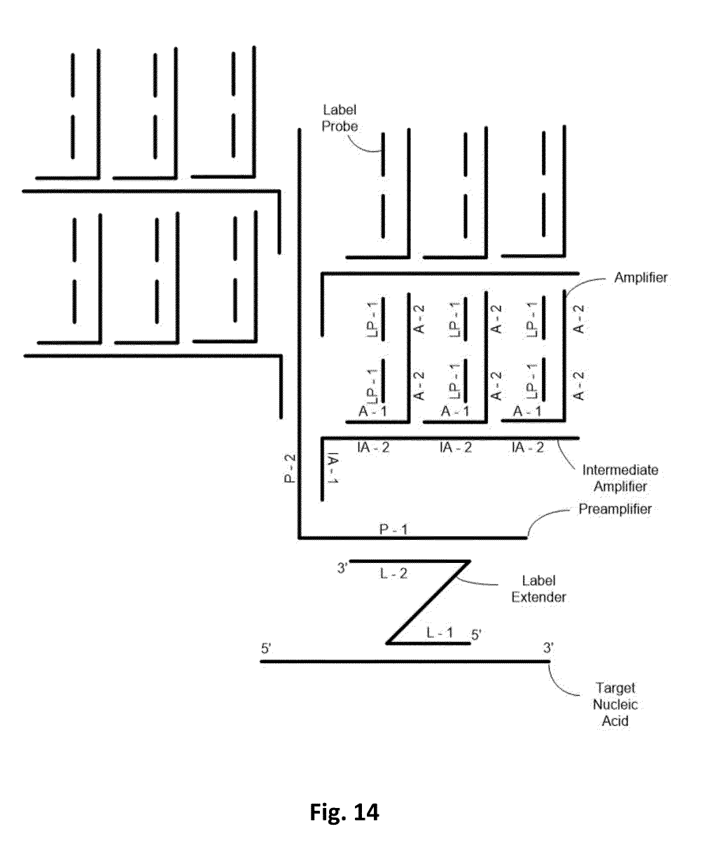

A “label probe system” comprises one or more polynucleotides that collectively comprise one or more labels and one or more polynucleotide sequences P-1 or A-1, each of which is capable of hybridizing to a label extender (e.g., at L-2). The label provides a signal, directly or indirectly. The amplifier (amplification multiplier) polynucleotide sequence A-1 is typically complementary to sequence L-2 in the label extenders, but can be complementary to the P-2 of a preamplifier (see, e.g., FIG. 10) or IA-2 of an intermediate amplifier (see, e.g., FIG. 14). The one or more polynucleotide sequences A-1 are optionally identical sequences or different sequences. The label probe system can include a plurality of label probes (e.g., a plurality of identical label probes or two or more sets of distinct label probes) and an amplification multimer; it optionally also includes a preamplifier, or optionally includes only label probes, for example. Preferably, the label probe system includes multiple copies of a label probe, amplification multimer (amplifier), at least one intermediate amplifier, and preamplifier, such as the non-limiting example depicted in FIG. 14. Other label probe systems may include multiple copies of a label probe, amplification multimer and preamplifier, but without an intermediate amplifier, such as the non-limiting example depicted in FIG. 10. The configuration of the label probe system within a particular embodiment is typically designed in the context of the overall assay, including factors such as the amount of signal required for reliable detection of the target analyte in the assay, the particular label being used, the number of label probes needed to provide the desired level of sensitivity, maintaining the desired specificity and sensitivity of the assay, and other factors known in the art.

A “preamplifier” is a polynucleotide that may serve as an intermediate between one or more label extenders and amplification multimers. For example, the preamplifier can be capable of hybridizing simultaneously to at least one label extender (preferably, one label extender) and to a plurality of amplification multimers. Preferably, the preamplifier is capable of hybridizing simultaneously to at least one label extender (preferably, one label extender) and to a plurality of intermediate amplifiers (of the same or different types), each of which is in turn capable of hybridizing to a plurality of amplification multimers (or to another intermediate amplifier). Preferably, a preamplifier is only capable of hybridizing simultaneously to a single label extender and to a plurality of amplification multimers (or intermediate amplifiers). The preamplifier can be, e.g., a linear or a branched nucleic acid. As noted for all polynucleotides, the preamplifier can include modified nucleotides and/or nonstandard internucleotide linkages as well as standard deoxyribonucleotides, ribonucleotides, and/or phosphodiester bonds.

An “intermediate amplifier” (IA) is a polynucleotide that serves as an intermediate between a preamplifier and amplification multimers. For example, the intermediate amplifier can be capable of hybridizing simultaneously to a preamplifier and to a plurality of amplification multimers (of the same or different types). As another example, the intermediate amplifier can be capable of hybridizing simultaneously to a preamplifier and to a plurality of copies of another intermediate amplifier. As noted for all polynucleotides, the intermediate amplifier can include modified nucleotides and/or nonstandard internucleotide linkages as well as standard deoxyribonucleotides, ribonucleotides, and/or phosphodiester bonds. The use of an intermediate amplifier is shown, e.g., in FIG. 14.

An “amplification multimer” (also referred to as “amplifier” herein) is a polynucleotide comprising a plurality of polynucleotide sequences A-2, typically (but not necessarily) identical polynucleotide sequences A-2. Polynucleotide sequence A-2 is complementary to a polynucleotide sequence in the label probe. The amplification multimer also includes at least one polynucleotide sequence that is capable of hybridizing to a label extender or to a nucleic acid that hybridizes or binds (directly or indirectly) to the label extender, e.g., a preamplifier or intermediate amplifier. For example, the amplification multimer optionally includes at least one polynucleotide sequence A-1; polynucleotide sequence A-1 is typically complementary to polynucleotide sequence L-2 of the label extenders. As another example, the amplification multimer optionally includes at least one polynucleotide sequence that is complementary to a polynucleotide sequence in a preamplifier (which in this example optionally includes at least one polynucleotide sequence A-1 that is complementary to polynucleotide sequence L-2 of the label extenders); see, e.g., the exemplary embodiment schematically illustrated in FIG. 10. As yet another example, the amplification multimer optionally includes at least one polynucleotide sequence that is complementary to a polynucleotide sequence in an intermediate amplifier (which in turn includes at least one polynucleotide sequence that is complementary to a polynucleotide sequence in a preamplifier that includes at least one polynucleotide sequence A-1 that is complementary to polynucleotide sequence L-2 of the label extenders; see FIG. 14). The amplification multimer can be, e.g., a linear or a branched nucleic acid. As noted for all polynucleotides, the amplification multimer can include modified nucleotides and/or nonstandard internucleotide linkages as well as standard deoxyribonucleotides, ribonucleotides, and/or phosphodiester bonds. Suitable amplification multimers are described, for example, in U.S. Pat. No. 5,635,352, U.S. Pat. No. 5,124,246, U.S. Pat. No. 5,710,264, and U.S. Pat. No. 5,849,481.

A “label probe” or “LP” is a single-stranded polynucleotide that comprises a label (or optionally that is configured to bind, directly or indirectly, to a label) that directly or indirectly provides a detectable signal. The label probe typically comprises a polynucleotide sequence that is complementary to the repeating polynucleotide sequence A-2 of the amplification multimer; however, if no amplification multimer is used in the bDNA assay, the label probe can, e.g., hybridize directly to a label extender.

A variety of additional terms are defined or otherwise characterized herein.

### DETAILED DESCRIPTION

In one aspect, the present invention provides methods for detecting target nucleic acids. The methods are particularly useful for detecting nucleic acids in situ and for detecting partially degraded or otherwise damaged nucleic acids. Compositions, kits, and systems related to or useful in practicing the methods are also described.

In certain aspects, the methods and compositions for detecting nucleic acids employ techniques and reagents similar to those employed in branched-chain DNA assays for detection of nucleic acids from solution. Accordingly, an overview of basic and multiplex branched-chain DNA assays is provided in the following section.

### Introduction to Branched-Chain DNA Assays

Branched-chain DNA (bDNA) signal amplification technology has been used, e.g., to detect and quantify mRNA transcripts from cell lines and to determine viral loads in blood. The basic bDNA assay is a sandwich nucleic acid hybridization procedure that enables direct measurement of mRNA expression, e.g., from crude cell lysate. It provides direct quantification of nucleic acid molecules at physiological levels. Several advantages of the technology distinguish it from other DNA/RNA amplification technologies, including linear amplification, good sensitivity and dynamic range, great precision and accuracy, simple sample preparation procedure, and reduced sample-to-sample variation.

In brief, in a typical bDNA sandwich assay for gene expression analysis (schematically illustrated in FIG. 1), a target mRNA whose expression is to be detected is released from cells and captured by a Capture Probe (CP) on a solid surface (e.g., a well of a microtiter plate) through synthetic oligonucleotide probes called Capture Extenders (CEs). Each capture extender has a first polynucleotide sequence that can hybridize to the target mRNA and a second polynucleotide sequence that can hybridize to the capture probe. Typically, two or more capture extenders are used. Probes of another type, called Label Extenders (LEs), hybridize to different sequences on the target mRNA and to sequences on an amplification multimer. Additionally, Blocking Probes (BPs), which hybridize to regions of the target mRNA not occupied by CEs or LEs, are often used to reduce non-specific target probe binding. A probe set for a given mRNA thus consists of CEs, LEs, and optionally BPs for the target mRNA. The CEs, LEs, and BPs are complementary to nonoverlapping sequences in the target mRNA, and are typically, but not necessarily, contiguous. Probe set design confers specificity for the given mRNA.

Signal amplification begins with the binding of the LEs to the target mRNA. An amplification multimer is then typically hybridized to the LEs. The amplification multimer has multiple copies of a sequence that is complementary to a label probe (it is worth noting that the amplification multimer is frequently, but not necessarily, a branched-chain nucleic acid; for example, the amplification multimer can be a branched, forked, or comb-like nucleic acid or a linear nucleic acid). A label, for example, alkaline phosphatase, is covalently attached to each label probe. (Alternatively, the label can be noncovalently bound to the label probes.) In the final step, labeled complexes are detected, e.g., by the alkaline phosphatase-mediated degradation of a chemilumigenic substrate, e.g., dioxetane. Luminescence is reported as relative light unit (RLUs) on a microplate reader. The amount of chemiluminescence is proportional to the level of mRNA expressed from the target gene.

In the preceding example, the amplification multimer and the label probes comprise a label probe system. In another example, the label probe system also comprises a preamplifier, e.g., as described in U.S. Pat. No. 5,635,352 and U.S. Pat. No. 5,681,697, which further amplifies the signal from a single target mRNA. In this example, the LEs hybridize to sequences on the target mRNA and to the preamplifier, the preamplifier has multiple copies of a sequence that is complementary to the amplification multimer, and the amplification multimer has multiple copies of a sequence that is complementary to the label probe. Like the amplification multimer, the preamplifier can be, e.g., a branched, forked, comb-like, or linear nucleic acid. In another example, the label probe system comprises one or more additional layers of amplifiers (called intermediate amplifiers herein) between the preamplifier and the amplification multimer, e.g., as described in U.S. patent application publication 2012/0003648. In yet another example, the label extenders hybridize directly to the label probes and no amplification multimer or preamplifier is used, so the signal from a single target mRNA molecule is only amplified by the number of distinct label extenders that hybridize to that mRNA.

Basic bDNA assays have been well described. See, e.g., U.S. Pat. No. 4,868,105 to Urdea et al. entitled “Solution phase nucleic acid sandwich assay”; U.S. Pat. No. 5,635,352 to Urdea et al. entitled “Solution phase nucleic acid sandwich assays having reduced background noise”; U.S. Pat. No. 5,681,697 to Urdea et al. entitled “Solution phase nucleic acid sandwich assays having reduced background noise and kits therefor”; U.S. Pat. No. 5,124,246 to Urdea et al. entitled “Nucleic acid multimers and amplified nucleic acid hybridization assays using same”; U.S. Pat. No. 5,624,802 to Urdea et al. entitled “Nucleic acid multimers and amplified nucleic acid hybridization assays using same”; U.S. Pat. No. 5,849,481 to Urdea et al. entitled “Nucleic acid hybridization assays employing large comb-type branched polynucleotides”; U.S. Pat. No. 5,710,264 to Urdea et al. entitled “Large comb type branched polynucleotides”; U.S. Pat. No. 5,594,118 to Urdea and Horn entitled “Modified N-4 nucleotides for use in amplified nucleic acid hybridization assays”; U.S. Pat. No. 5,093,232 to Urdea and Horn entitled “Nucleic acid probes”; U.S. Pat. No. 4,910,300 to Urdea and Horn entitled “Method for making nucleic acid probes”; U.S. Pat. No. 5,359,100; U.S. Pat. No. 5,571,670; U.S. Pat. No. 5,614,362; U.S. Pat. No. 6,235,465; U.S. Pat. No. 5,712,383; U.S. Pat. No. 5,747,244; U.S. Pat. No. 6,232,462; U.S. Pat. No. 5,681,702; U.S. Pat. No. 5,780,610; U.S. Pat. No. 5,780,227 to Sheridan et al. entitled “Oligonucleotide probe conjugated to a purified hydrophilic alkaline phosphatase and uses thereof”; U.S. patent application Publication No. US2002172950 by Kenny et al. entitled “Highly sensitive gene detection and localization using in situ branched-DNA hybridization”; Wang et al. (1997) “Regulation of insulin preRNA splicing by glucose” Proc Nat Acad Sci USA 94:4360-4365; Collins et al. (1998) “Branched DNA (bDNA) technology for direct quantification of nucleic acids: Design and performance” in Gene Quantification, F Ferre, ed.; Yao et al. (2004) “Multicenter Evaluation of the VERSANT Hepatitis B Virus DNA 3.0 Assay” J. Clin. Microbiol. 42:800-806; Elbeik et al. (2004) “Multicenter Evaluation of the Performance Characteristics of the Bayer VERSANT HCV RNA 3.0 Assay (bDNA)” J. Clin. Microbiol. 42:563-569; and Wilber and Urdea (1998) “Quantification of HCV RNA in clinical specimens by branched DNA (bDNA) technology” Methods in Molecular Medicine: Hepatitis C 19:71-78. In addition, kits for performing basic bDNA assays (QuantiGene® kits, comprising instructions and reagents such as amplification multimers, alkaline phosphatase labeled label probes, chemilumigenic substrate, capture probes immobilized on a solid support, and the like) are commercially available, e.g., from Affymetrix, Inc. (on the world wide web at www (dot) panomics (dot) com or www (dot) affymetrix (dot) com). Software for designing probe sets for a given mRNA target (i.e., for designing the regions of the CEs, LEs, and optionally BPs that are complementary to the target) is also available (e.g., ProbeDesigner™; see also Bushnell et al. (1999) “ProbeDesigner: for the design of probe sets for branched DNA (bDNA) signal amplification assays Bioinformatics 15:348-55).

It will be evident that, in a bDNA assay, the degree of signal amplification depends on factors such as the composition of the label probe system and the number of label extenders that hybridize to a given target molecule. For example, in a system in which signal amplification involves sequential hybridization of a preamplifier having twenty repeats to which the amplification multimer can hybridize and an amplification multimer having twenty repeats (sequence A-2) to which the label probe can bind, signal amplification is 400-fold per label extender (i.e., 400 copies of the LP are captured per LE). One of skill can choose a suitable degree of signal amplification for the desired application. Signal amplification can range, for example, from 400-fold to 5000-fold per label extender.

The basic bDNA assay described above generally permits detection of a single target nucleic acid per assay. Multiplex bDNA assays for detection of two or more targets simultaneously from solution have also been described. In brief, in an exemplary particle-based multiplex bDNA mRNA assay, different mRNAs are captured to different sets of microspheres. Each different mRNA is captured, through its own complementary set of CEs, to a distinguishable (e.g., fluorescently color-coded) set of microspheres bearing a CP complementary to that particular set of CEs. LEs and BPs are also hybridized to the mRNA targets, as for the singleplex assay described above. A label probe system (e.g., preamplifier, amplification multimer, and label probe) are then hybridized to the LEs as described above. Typically the label probe is fluorescently labeled (e.g., the LP can be biotinylated and detected with streptavidin conjugated phycoerythrin), and each set of microspheres is identified (e.g., by its unique fluorescence) and fluorescent emission by the label is measured for each set. The amount of label fluorescence for a given set of microspheres is proportional to the level of mRNA captured by that particular set of microspheres. A large number of mRNAs can be detected in a single reaction; for example, 50 or more targets can be assayed using 50 or more different sets of microspheres.

For additional information relevant to multiplex sandwich assays for detection of nucleic acids from solution, see U.S. Pat. Nos. 7,803,541 and 8,426,578 “Multiplex branched-chain DNA assays” to Luo et al., U.S. Pat. Nos. 8,628,918 and 8,632,970 “Multiplex capture of nucleic acids” to Luo et al., and U.S. Pat. No. 7,709,198 “Multiplex detection of nucleic acids” to Luo et al. See also Flagella et al. (2006) “A multiplex branched DNA assay for parallel quantitative gene expression profiling” Anal. Biochem. 352:50-60 and U.S. Pat. Nos. 8,114,681 and 8,685,753 “Highly multiplexed particle-based assays” to Martin, et al. QuantiGene® Plex kits for performing basic multiplex bDNA assays comprising instructions and reagents such as preamplifiers, amplification multimers, label probes, capture probes immobilized on microspheres, and the like are commercially available, e.g., from Affymetrix, Inc, as are ViewRNA™ kits for performing in situ bDNA assays.

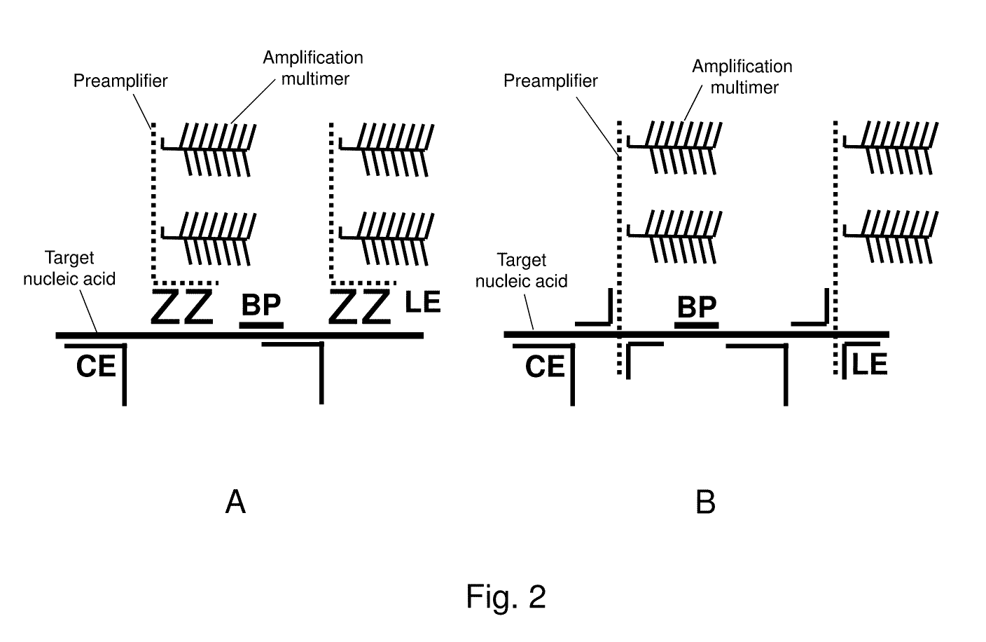

In such sandwich bDNA assays, requiring binding of two label extenders to a component of the label probe system to capture the label probe system (e.g., a single preamplifier that hybridizes with both of the two label extenders) to the target nucleic acid can increase specificity of the specific label probe system at issue (e.g., the specificity of a particular preamplifier with the intended target nucleic acid). FIG. 2 schematically illustrates two configurations in which binding of two label extenders to a preamplifier is required to capture the preamplifier to the target nucleic acid. FIG. 2 Panel A illustrates a “ZZ” configuration of the label extenders, where the 5′ end of both label extenders binds to the target nucleic acid while the 3′ end of both label extenders binds to the preamplifier (or vice versa). FIG. 2 Panel B illustrates a cruciform configuration of the label extenders, where for one label extender the 5′ end binds to the target nucleic acid while the 3′ end binds to the preamplifier and for the other label extender the 3′ end binds to the target nucleic acid while the 5′ end binds to the preamplifier. See, e.g., US patent application publication 2007/0015188 and U.S. Pat. No. 5,635,352.

### In Situ Detection of Nucleic Acids

Detection of nucleic acids in situ presents different challenges than do solution-based assays such as those described above. For example, tissue and cell samples for in situ analysis are typically fixed (to preserve the tissue or cells by, e.g., stopping cellular activity and maintaining morphology) and then pretreated to unmask the target nucleic acids (to permit access to nucleic acids by probes). Unmasking generally involves uncross-linking ionic and covalent bonds between the target nucleic acids and adjacent proteins (e.g., by heating in a mild acidic or alkaline solution) and permeabilization (e.g., by protease treatment). In many situations where in situ analysis is desired over an assay that simply lyses the cells of the sample, the morphological features of the cells/tissue of the sample are important for the analysis as, e.g., identifying the particular cells and cell types that are expressing a particular gene (and at what level of expression) can be important for various research and clinical analyses of cancers and other diseases and conditions. Thus, steps such as fixation, uncross-linking, and permeabilization have to be controlled in order to avoid undesired damage to the morphological features that will be observed during analysis. Such control, however, can limit sample preparation steps and hinder subsequent detection of nucleic acids (e.g., increased permeabilization allows easier and more complete access of probes to nucleic acids within the cells, but must be balanced against destroying the cellular morphology that will play a part of the analysis).

In in situ assays, binding of various proteins to the target nucleic acids and/or crosslinking of proteins to the target nucleic acids during fixation can hinder access to the nucleic acids by labeled probes (or, in a bDNA assay, by the label extenders and subsequent components of the employed label probe system). Variation in tissue fixation regimens leads to over-crosslinking of some samples, exacerbating this problem. Extended incubation of the sample in fixative tends to produce over-crosslinking. Too short an incubation in the fixative, however, can result in undesired degradation of RNA targets, since fixation inactivates many cellular activities including RNases.

Similarly, because over-pretreatment will ruin the cellular morphology (e.g., via excessive heating and acid or base treatment to uncross-link or via over-permeabilization), some samples are under-pretreated. Although under-pretreatment ensures that more of the cellular morphology is retained, more proteins also remain cross-linked to the nucleic acids and more of the proteins that were already bound to the nucleic acids when the sample was fixed (in formalin or another fixative) will still be present and masking potential label extender binding sites.

As another example, many samples on which in situ analysis is to be performed are poorly and inconsistently preserved via fixation, especially samples from human patients that are taken at a variety of healthcare and laboratory facilities for subsequent analysis that often occurs at a different facility (e.g., a separate laboratory in compliance with the Clinical Laboratory Improvement Amendments of 1988 that employs a specific diagnostic test). RNA targets, in particular, are often subject to undesirable degradation and fragmentation and/or to over-crosslinking resulting from over-fixation and can present difficulties when not only detection of expression but also accurate measurement of the level of expression is required or at least desirable for a particular gene or genes in the context of an associated disease or condition.

Attempting to automate in situ analysis can introduce additional difficulties, for example, due to incomplete removal of unbound materials at various stages of the process. While in situ analysis when performed manually routinely involves agitation of the sample during washes, automated platforms typically perform washes without agitation (for example, by gravity and/or fan- or vacuum-assisted draining of one solution and its replacement by fresh solution). Additionally, automated in situ analysis will generally employ the same treatments for all of the samples being analyzed at a particular time (e.g., all of the samples being processed with the instrument(s) during a single assay run), and this can cause additional difficulties when there is variation between samples with respect to nucleic acid quality, quantity, preservation and other factors that would be individually accounted for during manual in situ analysis by customization of assay protocols to account for individual sample deficiencies.

The present invention provides methods that address such challenges, facilitating the robust detection of nucleic acids in situ, even from highly cross-linked, poorly prepared, and degraded samples handled by automated platforms. Gentle pretreatment regimens that better maintain cell and tissue morphology can be employed. Detection can be performed, e.g., with samples on slides or with alternative approaches such as ISH-based flow cytometry. Single RNA transcripts or other single or low copy number targets can be detected. While currently the pretreatment protocol has to be optimized for the sample at issue (considering factors such as the sample's type of tissue, type of cells, age, method of fixation, etc.), the instant methods are more robust and are suitable for a one-size-fits-all pretreatment regimen, which is especially useful for automation but also for manual processing. The methods facilitate detection of nucleic acids in situ by increasing sensitivity of detection, which work in our laboratory has identified as being surprisingly more crucial to successful in situ detection than specificity of probe binding as previous approaches were discovered to suffer more from false negatives than false positives, and the loss of specificity from increasing the sensitivity of the assay was significantly less than what would ordinarily be expected.

As noted above, in sandwich hybridization assays for nucleic acids captured from solution, requiring that two label extenders bind simultaneously to a component of the label probe system (e.g., a preamplifier) to capture the label probe system to the target nucleic acid can increase specificity. In in situ assays, in contrast, requiring that two label extenders bind simultaneously to the label probe system component can interfere with successful detection of the target nucleic acid. Configuring the label extenders and label probe system such that a preamplifier (or other component of the label probe system) hybridizes to a single label extender is therefore typically preferable for in situ detection, as sensitivity has unexpectedly been shown to be a significantly more limiting factor on successful assay performance than specificity.

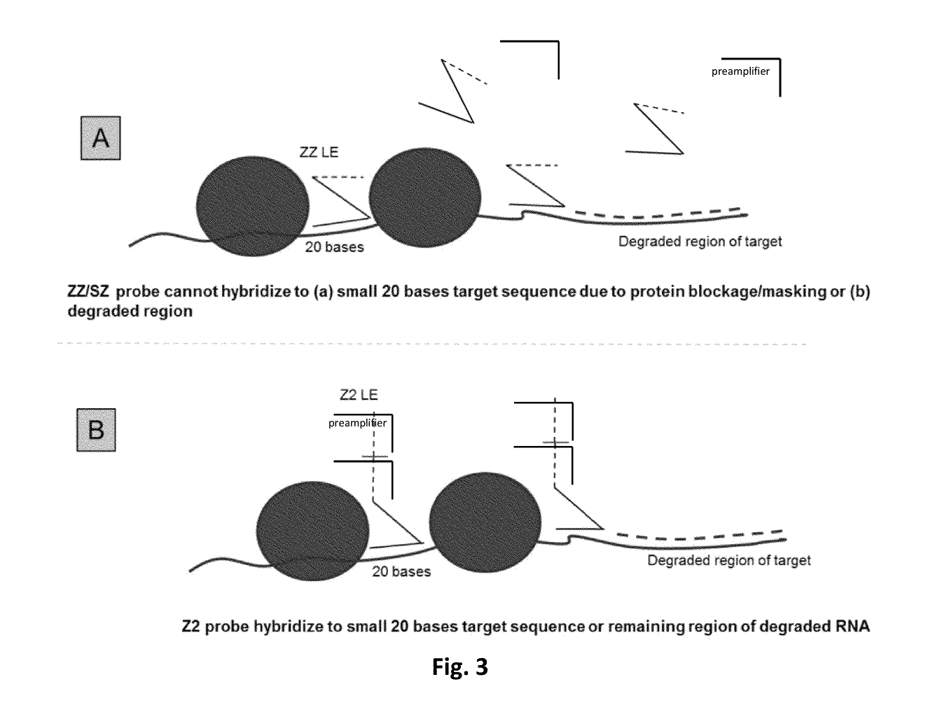

FIG. 3 Panels A and B contrast binding of label extenders to a target RNA and a preamplifier when the preamplifier hybridizes to two label extenders (Panel A) versus a single label extender (Panel B). In this illustration, each label extender is complementary to 20 nucleotides of the target RNA. As shown in FIG. 3, requiring that two label extenders bind to the target nucleic acid and to the label probe system component effectively doubles the length of accessible, intact target region that is required for any preamplifier(s) to be associated with that target region. Proteins bound or cross-linked to the target RNA prevent complete unmasking of the RNA. In the configuration shown in Panel A, incomplete unmasking of the RNA means that some accessible regions of the RNA are too short to permit binding of both label extenders, and, since binding of a single label extender is insufficient to capture the preamplifier to the target RNA, the label probe system is not captured to the target (Panel A, left-hand side). In the configuration shown in Panel B, binding of a single label extender can still capture the preamplifier (even multiple copies of the preamplifier) to the target RNA (Panel B, left-hand side). Similarly, degradation of the RNA means that some regions to which label extenders would otherwise hybridize are not available. Such degradation interferes more severely with capture of the preamplifier in configurations where two label extenders are required to bind to the target and preamplifier (Panel A, right-hand side) than configurations in which each preamplifier binds to only a single label extender (Panel B, right-hand side). Configuring the label extenders such that each copy of the preamplifier binds to a single label extender, rather than to two or more label extenders simultaneously, therefore facilitates capture of the preamplifier (and therefore the remainder of the label probe system) to the target nucleic acid and resulting detection of the nucleic acid.

As an additional advantage, configuring the preamplifier (or other label probe system component) to hybridize to a single label extender instead of requiring binding to two of the label extenders increases the maximum number of preamplifier copies that can be bound to the target nucleic acid (and therefore increases the maximum number of copies of the label probe and label that are ultimately captured to the target, and therefore the resulting maximum signal strength). For example, assuming that all label extender binding sites on the target are available in an ideal scenario, if 40 label extenders are employed, 40 preamplifiers can theoretically be captured where each preamplifier hybridizes to a single label extender and each label extender hybridizes to a single preamplifier, while only 20 preamplifiers can theoretically be captured where each preamplifier hybridizes to two of the label extenders. Since twice as many preamplifiers are captured to the target nucleic acid, ultimately twice as many label probes and thus copies of the label are also captured to the target; signal strength is thus doubled. It is important to note, however, that as discussed above, portions of the target nucleic acid will often be improperly and/or incompletely unmasked, or will simply be degraded, and thus even if a set of 40 label extenders is designed for a particular target nucleic acid, it will be extremely rare for all 40 to be able to hybridize to a corresponding target nucleic acid in the sample. Thus, in many cases, the increase in signal is actually greater than the theoretical conversion would indicate. For instance, if a particular target RNA has proteins bound to it and includes some degraded regions such that only 30 of the 40 designed label extenders are able to bind, in a best case scenario the approach of using 2 label extenders per preamplifier would lead to 15 preamplifiers bound to the target, while the approach of using 1 label extender per preamplifier would lead to 30 preamplifiers bound to the target. However, it is unlikely that the loss of accessibility to 10 of the label extenders to the target region would occur in such a sequence and arrangement such that 15 pairs of label extender/preamplifier sites would remain. Rather, it would be expected that at least some of the non-accessible regions on the target nucleic acid would affect only one member of a label extender pair, and thus the 2 label extender per preamplifier approach would result in less than 15 preamplifiers bound to the target. As another example, if a target mRNA is masked by proteins, lipids, and other cellular materials and/or is partially degraded such that hybridization efficiency for a single oligonucleotide label extender is 20%, in embodiments in which binding of two label extenders is required to capture the preamplifier, theoretical hybridization efficiency for the pair of label extenders/preamplifier would be 4% (0.2×0.2=0.04): five-fold less than in embodiments in which binding of a single label extender captures the preamplifier. For a typical mRNA transcript, where binding of 12-20 preamplifiers is desirable for reliable detection, the overall difference in hybridization efficiency between these embodiments is 5×(12 to 20): 60-100-fold.

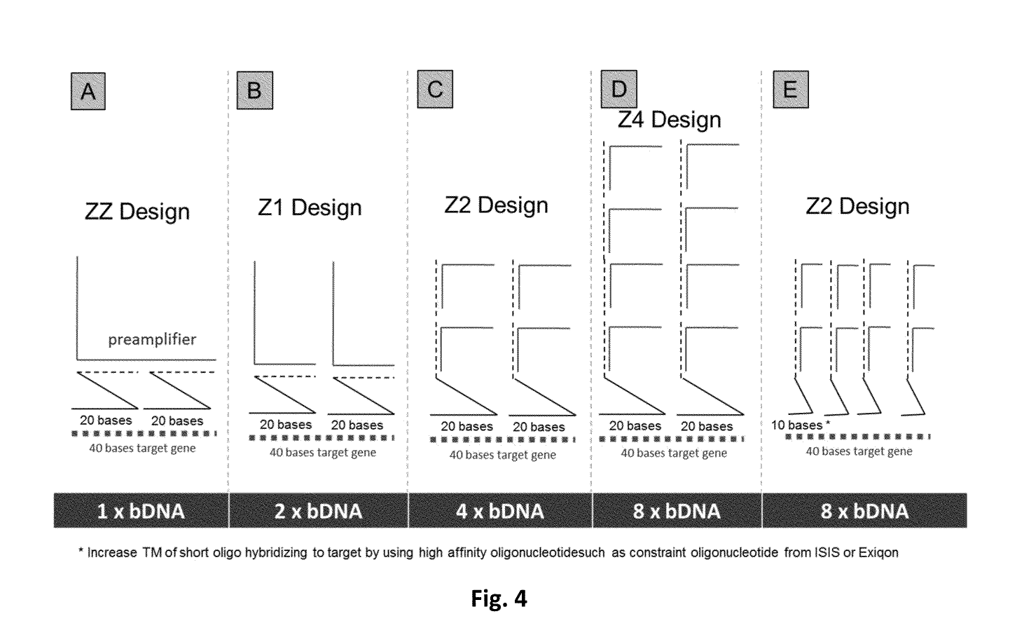

Signal strength can also be increased by configuring each label extender to hybridize to two or more copies of the preamplifier (or other component of the label probe system), e.g., to three or more or four or more copies of the preamplifier. FIG. 4 Panels A-E compare the number of preamplifiers (or other component of the label probe system) that can be captured to 40 bases of a target nucleic acid, in an embodiment in which two label extenders that each hybridize to 20 nucleotides of the target are required for capture of one preamplifier (ZZ LEs, Panel A) with embodiments in which two label extenders each hybridize to 20 nucleotides of the target and to a single preamplifier (Z1 LE, Panel B), two label extenders each hybridize to 20 nucleotides of the target and to two preamplifiers (Z2 LEs, Panel C), two label extenders each hybridize to 20 nucleotides of the target and to four preamplifiers (Z4 LEs, Panel D), and four label extenders each hybridize to 10 nucleotides of the target and to two preamplifiers (Z2 LEs, Panel E). The number of preamplifiers captured is noted under each panel as the number “x bDNA” for convenience of comparison.

Signal strength can also be increased by increasing the number of label extenders that hybridize to the target nucleic acid, thereby increasing the number of copies of preamplifier, amplification multimer, label probe, and so on that are captured to the target nucleic acid. Essentially any desired number of label extenders can be employed, for example, 1-40 or more label extenders. Preferably, at least 5, at least 10, at least 15, at least 20, at least 25, at least 30, at least 35, at least 40, at least 45 or at least 50 label extenders hybridize to the target nucleic acid. Each label extender typically includes a polynucleotide sequence L-1 that is complementary to a polynucleotide sequence in the target nucleic acid. To ensure stable hybridization of the label extender to the target nucleic acid, the length of L-1 has typically been selected such that the Tₘ of the label extender-target complex under the assay conditions is greater than the hybridization temperature. L-1 can be of essentially any convenient length, e.g., 20-40 nucleotides, 20-30 nucleotides or 20-25 nucleotides. Where the number of label extenders that can be employed is limited, for example, by the length of the target nucleic acid or a distinguishing region thereof, shorter L-1 sequences can be employed. Similarly, where incomplete unmasking and/or degradation of the target limit the availability of the target's full-length for hybridization, shorter L-1 sequences can be employed to help ensure that a greater number of label extenders will actually hybridize with the target. For example, L-1 can be 20 nucleotides or less, e.g., 5-20 or 10-20 nucleotides. In one class of embodiments, L-1 is less than 15 nucleotides in length. For example, L-1 can be 5-14, 5-13, 7-13, 7-10, or 5-10 nucleotides in length.

To achieve stable hybridization of a label extender having a short L-1 sequence to the target nucleic acid, one or more non-natural nucleotides can be incorporated into the label extender (in particular, into L-1). Suitable non-natural nucleotides for increasing the Tₘ of short probes include, e.g., locked nucleic acid analogs and constrained ethyl analogs. See, e.g., SantaLucia Jr. (1998) Proc Natl Acad Sci 95:1460-1465, U.S. Pat. No. 6,001,983, U.S. Pat. No. 6,037,120, U.S. Pat. No. 6,140,496, U.S. Pat. No. 7,572,582, U.S. Pat. No. 6,670,461, U.S. Pat. No. 6,794,499, U.S. Pat. No. 7,034,133, U.S. Pat. No. 5,700,637, U.S. Pat. No. 5,436,327, U.S. Pat. No. 7,399,845, U.S. Pat. No. 7,569,686, U.S. Pat. No. 7,741,457, and U.S. Pat. No. 8,022,193. Appropriate combinations of probe Tₘ and stringency of hybridization to achieve stable hybridization of the label extender to the target (and of components of the label probe system to the label extender and to each other) can, for example, be determined experimentally by one of skill in the art. Stringency can be controlled, for example, by controlling the formamide concentration, chaotropic salt concentration, salt concentration, pH, organic solvent content, and/or hybridization temperature.

As noted, the Tₘ of any nucleic acid duplex can be directly measured, using techniques well known in the art. For example, a thermal denaturation curve can be obtained for the duplex, the midpoint of which corresponds to the Tₘ. It will be evident that such denaturation curves can be obtained under conditions having essentially any relevant pH, salt concentration, solvent content, and/or the like.

The Tₘ for a particular duplex (e.g., an approximate Tₘ) can also be calculated. For example, the Tₘ for an oligonucleotide-target duplex can be estimated using the following algorithm, which incorporates nearest neighbor thermodynamic parameters: Tm (Kelvin)=ΔH°/(ΔS°+R ln Cₜ), where the changes in standard enthalpy)(ΔH° and entropy (ΔS°) are calculated from nearest neighbor thermodynamic parameters (see, e.g., SantaLucia (1998) “A unified view of polymer, dumbbell, and oligonucleotide DNA nearest-neighbor thermodynamics” Proc. Natl. Acad. Sci. USA 95:1460-1465, Sugimoto et al. (1996) “Improved thermodynamic parameters and helix initiation factor to predict stability of DNA duplexes” Nucleic Acids Research 24: 4501-4505, Sugimoto et al. (1995) “Thermodynamic parameters to predict stability of RNA/DNA hybrid duplexes” Biochemistry 34:11211-11216, and et al. (1998) “Thermodynamic parameters for an expanded nearest-neighbor model for formation of RNA duplexes with Watson-Crick base pairs” Biochemistry 37: 14719-14735), R is the ideal gas constant (1.987 cal·K⁻¹ mole⁻¹), and Cₜ is the molar concentration of the oligonucleotide. The calculated Tₘ is optionally corrected for salt concentration, e.g., Na⁺ concentration, using the formula 1/Tₘ(Na⁺)=1/Tₘ(1M)+(4.29f (G·C)−3.95)×10⁻⁵ ln [Na⁺]+9.40×10⁻⁶ ln²[Na⁺]. See, e.g., Owczarzy et al. (2004) “Effects of Sodium Ions on DNA Duplex Oligomers: Improved Predictions of Melting Temperatures” Biochemistry 43:3537-3554 for further details. A Web calculator for estimating Tm using the above algorithms is available on the Internet at scitools.idtdna.com/analyzer/oligocalc.asp. Other algorithms for calculating Tₘ are known in the art and are optionally applied to the present invention.

Employing a short L-1 sequence can in some instances result in increased binding of a label extender (e.g., from a set of label extenders designed for a particular target) to non-target sequences. This nonspecific binding can be tolerated, however, in embodiments in which it does not unacceptably increase signal background and the particular embodiment's signal to noise (background) ratio is maintained at acceptable levels. For example, in embodiments in which a chromogenic signal system is employed (e.g., an enzymatic label and a chromogenic substrate detected by bright-field light microscopy), binding of multiple label extenders and their associated label probe system components close together in a single area is required to produce a detectable colored spot (e.g., a spot that can be observed via bright-field microscopy). Thus, for many nucleic acids where the region targeted for the assay may be, e.g., 50, 100, 250, 500 or more bases, there will be multiple label extenders designed for the targeted region, and thus occasional nonspecific binding of single label extenders to non-target regions will therefore not result in detectable spots and can be tolerated and/or easily disregarded as non-specific signal in contrast to the stronger signal produced from multiple label extenders and their associated label probe systems hybridizing within the targeted region(s) of the nucleic acid target. The concentration of the label extenders is optionally reduced to help ensure specificity. For example, in embodiments in which each label extender hybridizes to one preamplifier and each preamplifier hybridizes to a single label extender, the concentration of the label extenders can be reduced about 10-30-fold compared to embodiments in which two label extenders are required to capture each preamplifier.

Different label extenders typically hybridize to non-overlapping sequences in the target nucleic acid. The label extenders can, but necessarily, hybridize to contiguous polynucleotide sequences of the target nucleic acid. In one class of embodiments, the label extenders hybridize to noncontiguous polynucleotide sequences of the target nucleic acid, e.g., separated by 1-20 or more nucleotides, e.g., 10-20 nucleotides. The label extenders can, but need not be, contiguous with any capture extenders and/or blocking probes employed. All of the label extenders hybridizing to a given target nucleic acid typically include an identical sequence L-2. Optionally, however, different label extenders include different sequences L-2.

At any of various steps, materials not captured to the target nucleic acid are optionally separated from the target nucleic acid. For example, after the label extenders are hybridized to the target nucleic acid, the sample is optionally washed to remove unbound label extenders; after the preamplifier is hybridized to the label extenders, the sample is optionally washed to remove unbound preamplifier; after the intermediate amplifier is hybridized to the preamplifier, the sample is optionally washed to remove unbound intermediate amplifier; after the amplification multimer is hybridized to the intermediate amplifier, the sample is optionally washed to remove unbound amplification multimer; and/or after the label probe is hybridized to the amplification multimer, the sample is optionally washed to remove unbound label probe prior to detection of the label. Various hybridizations (and their associated wash steps) can be performed simultaneously or sequentially, as desired.

In situ detection can be conveniently automated using commercially available platforms, for example, the BOND-III and BOND RX systems from Leica Biosystems (www (dot) leicabiosystems (dot) com). The sensitivity achieved by such automated systems, however, tends to be lower than that achieved by manual sample processing. Work in our laboratory indicates that sensitivity is largely limited by incomplete removal of unbound material at various stages of the procedure. Manual assays generally involve prolonged agitation of the sample in a buffered wash solution, while automated platforms remove one solution, e.g., through gravity and/or fan- or vacuum-assisted draining, and replace it with fresh solution without any agitation of the sample. In addition, automated platforms typically employ a smaller volume of wash solution than do manual assays. Volumes of wash (and other) solutions employed in automated platforms may be preset and difficult for a user of the system to adjust in order to compensate for the characteristics of a particular sample, as is commonly done in manual assays. Automated platforms typically also employ a single set of washing conditions (e.g., a single type of wash solution) for all samples, whereas washing conditions may be optimized for individual samples (or sample types) in manual assays. The techniques described herein for increasing signal strength overcome these drawbacks and facilitate automated detection of targets in situ.

The methods can optionally be used to quantitate the amount of the target nucleic acid that is present in the sample. For example, in one class of embodiments, an intensity of a signal from the label is measured and correlated with a quantity of the target nucleic acid present. Optionally, both localization and quantitation are performed (e.g., to determine the amount of the target nucleic acid present in a particular tissue, cell type, organelle, subcellular location, or the like).

It will be evident that the techniques described herein can be used in essentially any combination as desired. For example, in situ detection of a target nucleic acid can be performed using one or more label extenders, a preamplifier that hybridizes to a single label extender, an amplification multimer, and a label probe, where each label extender hybridizes to two or more copies of the preamplifier and has an L-1 sequence of less than 15 nucleotides. The label probe system can also include one or more intermediate amplifiers. As noted, such techniques can detect even short (e.g., less than 400 nucleotides or 25 nucleotides or less) target nucleic acids or target regions, suboptimally processed target nucleic acids (e.g., from over cross-linked samples), and/or poorly preserved or partially degraded target nucleic acids (e.g., RNAs from formalin-fixed, paraffin-embedded tissue sections or samples, including such samples that have been in long-term storage) after gentle pretreatment (e.g., as described below), and can be automated (e.g., on platforms that do not provide agitation during washes to remove unbound materials). The label can be an enzyme that acts on a chromogenic substrate, e.g., in embodiments in which manual or automated in situ detection of a target in cells or tissues on slides is performed. As another example, the label can be a fluorescent label, e.g., in embodiments in which in situ detection of a target in cells is performed by flow cytometry or another technique where the fluorescent detector has a short exposure time to the target or where the detector is less sensitive, and therefore greater signal is desirable.

As noted above, the techniques can be employed in detection of short target nucleic acids, e.g., nucleic acids having less than 400 nucleotides, e.g., target nucleic acids 300 nucleotides or less, 200 nucleotides or less, 100 nucleotides or less, 50 nucleotides or less, or even 25 nucleotides or less in length. The techniques can also be employed in detection of a target nucleic acid that comprises a short target region, where the target region is the region of the target nucleic acid within which all of the label extenders that were designed to be complementary to that target hybridize. For example, the target region can have less than 400 nucleotides. For example, the target region can be 300 nucleotides or less, 200 nucleotides or less, 100 nucleotides or less, 50 nucleotides or less, or even 25 nucleotides or less in length. The full-length target nucleic acid is optionally longer than the target region within which the label extenders hybridize. The target region can be a portion of the overall nucleic acid that can be used to distinguish it from other, non-target nucleic acids present or suspected to be present in the sample. For example, a target region can be used to distinguish one variant of a gene transcript relative to another variant of the same gene.

In one exemplary class of embodiments in which the target-specific region of a target (compared to its related variants) is small, the techniques can be employed for detection and distinguishing of IgG4 as compared to IgG where the relevant region of a target nucleic acid for design of the set of label extenders is small (e.g., a conserved target region that is unique or has minimal overlap within the organism of interest). For detection of IgG, almost 1 kb is available as a target region for detection of the transcript. Given that IgG4 is a subtype of IgG, however, a much smaller target region is specific for detection of IgG4. See, e.g., U.S. application Ser. No. 14/622,219 filed Feb. 13, 2015 and entitled “Methods for Diagnosing IgG4-Related Disease,” which describes detection of IgG4 using four label extenders complementary to the IgG4-specific target region and determination of the ratio of cells expressing IgG4 versus IgG. The techniques herein can be employed in conjunction with those described in U.S. application Ser. No. 14/622,219 or in similar situations, e.g., to count cells expressing particular genes, including situations where a particular target only presents a small target region for accurate interrogation as compared to other similar nucleic acids that are or may be present within the sample to be assayed.

As another example of how the techniques can be employed in detection of a target nucleic acid containing a short polynucleotide sequence that distinguishes it from other, non-target nucleic acids suspected to be in the sample, a splice junction in an alternatively spliced mRNA or a translocation site on a chromosome can be detected. In one exemplary class of embodiments, a label extender having L-1 complementary to a polynucleotide sequence spanning a splice junction or translocation site in the target nucleic acid is employed. A preamplifier that binds to a single label extender is also employed. Preferably the label extender binds to two or more preamplifiers.

The techniques are suitable for use even in situations where expression levels of genes vary greatly, e.g., depending on cell type, growth state, presence of disease, or the like. Expression levels of a target nucleic acid, e.g., a target RNA, can vary widely from sample to sample and/or even within a particular sample based on the tissue or cell types present. An ideal assay for nucleic acid detection can thus detect both the high and the low ends of the expression range. The techniques described herein fulfill this and other needs. For example, the techniques described herein for in situ detection can be employed in conjunction with the techniques described in U.S. application Ser. No. 14/634,108, filed Feb. 27, 2015 and entitled “Diagnosis of Multiple Myeloma and Non-Hodgkin Lymphoma” to assess expression for one or more genes in different cell types (e.g., plasma cells and B-lymphocytes) within the same sample (e.g., tissue sample from a lymph node); plasma cells have significantly higher express of IgK and IgL as compared to B lymphocytes to be assayed for multiple myeloma and non-Hodgkin lymphoma diagnostic attempts. The gentler pretreatment regimens described herein for preserving morphology can be particularly useful in such embodiments, where the diagnostic call is based on not only the level of expression but also on which cells are showing certain levels of expression.

The techniques can be employed in conjunction with techniques for detection of one or more proteins, e.g., immunohistochemistry (IHC). IHC can be performed simultaneously or sequentially with in situ nucleic acid detection as described herein. In other embodiments, the techniques for nucleic acid detection are useful for detecting expression where a protein of interest is secreted and IHC would thus not be able to assess whether the cells themselves are expressing the relevant mRNA. For example, the techniques described herein can be used in conjunction with those described in U.S. application Ser. No. 14/616,297 filed Feb. 6, 2015 and entitled “Differential Diagnosis of Hepatic Neoplasms,” to assess expression of albumin mRNA in situ when the protein is secreted. Again, the gentler pretreatment regimens described herein for preserving morphology can be useful in such embodiments, where diagnosis depends not only the level of expression but also on the tumor architecture.

It will be evident that, although the preceding techniques have been described in the context of in situ nucleic acid detection, the techniques can also be employed in solution-based sandwich assays. Solution-based sandwich assays can also benefit from the preceding embodiments in various situations, such as when a very low number of copies of a target is present within the sample (e.g., due to low expression of a target RNA, or having a heterogeneous sample where a particular target such as an mRNA may not be present in all cells), and also when there is a very small quantity of cells available for the assay (as is common with many clinical samples taken from patients when a single sample must be used for multiple tests). For example, a target nucleic acid can be captured from solution to a solid support (e.g., through hybridization of one or more capture extenders to the target nucleic acid and to a support-bound capture probe) and then detected through binding of one or more label extenders, a preamplifier that hybridizes to a single label extender, an amplification multimer, and a label probe. The label probe system can also include one or more intermediate amplifiers. Optionally, each label extender has an L-1 sequence of less than 15 nucleotides, e.g., for detection of short target nucleic acids (e.g., 25 nucleotides or less, e.g., microRNA). Optionally, each label extender hybridizes to two or more preamplifiers. Suitable solid supports are known in the art and include, but are not limited to, particles, slides, and multiwell plates.

The techniques described herein can also be employed in multiplex assays for simultaneous detection of two or more nucleic acids, if desired. For example, in sandwich or in situ assays, a different label probe system comprising a detectably different label can be bound to the label extenders that hybridize to each different target nucleic acid. (For example, a first set of label extenders that hybridize to a first target nucleic acid include a first sequence L-2 that is complementary to a first sequence A-1 in a first label probe system, while a second set of label extenders that hybridize to a second target nucleic acid include a second sequence L-2 (different from the first sequence L-2) that is complementary to a second sequence A-1 in a second label probe system; the second label probe system includes a label that is detectably different from the label in the first label probe system, and the components of the second label probe system are not expected to cross-hybridize with those of the first label probe system.) In sandwich assays, different target nucleic acids can be captured to different subsets of particles (e.g., having different fluorescent emission spectra, different optical barcodes, and/or different sizes) or to different positions on a spatially addressable solid support, e.g., as described in U.S. Pat. No. 7,803,541 (A-1 referred to therein as “M-1”).

The methods can be used to detect the presence of target nucleic acids in essentially any type of sample. For example, the sample can be derived from an animal, a human, a plant, a cultured cell, a virus, a bacterium, a pathogen, and/or a microorganism. The sample optionally includes a cell lysate, an intercellular fluid, a bodily fluid (including, but not limited to, blood, serum, saliva, urine, sputum, or spinal fluid), and/or a conditioned culture medium, and is optionally derived from a tissue (e.g., a whole tissue, a tissue section, or a tissue homogenate), a biopsy, and/or a tumor. The sample can include cells immobilized on a solid support, e.g., a microscope slide. As a few examples, the methods can be used for in situ detection of target nucleic acid in formalin-fixed paraffin embedded material (e.g., biopsy or tissue samples), fresh frozen tissue sections, fine needle aspirate biopsies, tissue microarrays, cellular samples (e.g., cells isolated from blood (including whole blood), bone marrow or sputum, e.g., samples prepared using centrifugation or smeared on a slide), blood smears on slides (including whole blood smears), cells from a mass (e.g., a soft tissue mass), and other sample types, e.g., where the cellular morphology is sufficiently intact to allow the identification of the cells of interest.

Similarly, target nucleic acids can be essentially any desired nucleic acids. As just a few examples, target nucleic acids can be derived from one or more of an animal, a human, a plant, a cultured cell, a microorganism, a virus, a bacterium, or a pathogen. A target nucleic acid can be, e.g., a DNA (e.g., a chromosome) or an RNA (e.g., an mRNA, a microRNA, a pri-miRNA, or a pre-miRNA).

The methods can be used for gene expression analysis. Accordingly, in one class of embodiments, the target nucleic acid is an mRNA. The methods can also be used for clinical diagnosis and/or detection of microorganisms, e.g., pathogens. Thus, in certain embodiments, the nucleic acids include bacterial and/or viral genomic RNA and/or DNA (double-stranded or single-stranded), plasmid or other extra-genomic DNA, or other nucleic acids derived from microorganisms (pathogenic or otherwise). It will be evident that double-stranded target nucleic acids will typically be denatured before hybridization with label extenders and the like.

### Gentle Pretreatment Regimens

In situ hybridization generally involves several stages, including, e.g., sample collection, fixation, dewaxing (or deparaffinization), unmasking (e.g., heat and proteinase treatments), denaturation of double-stranded target nucleic acids, and/or intentional degradation of cellular RNA (for detection of DNA targets), as well as hybridization of the probes employed for detection of the targets. For example, a typical workflow for preparation of an FFPE sample for in situ hybridization begins with fixation of the sample to preserve tissue morphology and inactivate cell activity, e.g., by placing tissue less than 3 mm thick into 20 volumes of 10% neutral buffered formalin (NBF) for 16-24 hours. Formalin crosslinks proteins and nucleic acids via covalent bonds. (Other fixatives are known in the art, e.g., paraformaldehyde.) The extent of crosslinking varies, e.g., with the amount of fixative and fixation time employed. The fixed tissue sample is embedded in wax, sectioned, and mounted and baked onto a slide. The mounted sections are dewaxed (e.g., by heat and treatment with Histo-Clear or by treatment with xylene), rehydrated (e.g., in an ethanol solution or in a series of washes with decreasing concentrations of ethanol), and optionally dried (e.g., brief air drying at room temperature). The sample is then pretreated to unmask the target nucleic acid(s). Pretreatment typically includes heating the sample in a mild acidic or alkaline solution at 90-100° C. to uncross-link covalent bonds, e.g., between the target nucleic acid and adjacent proteins, and treating with protease to permeabilize the tissue or cells. Pretreatment is typically followed by a brief post-fixation step (e.g., 5 minutes in 4% formaldehyde or paraformaldehyde) to ensure that the nucleic acid molecules do not diffuse out of the cells during subsequent steps and to inactivate the protease.

As detailed above, variation in sample preparation techniques can present challenges for in situ detection. Extensive fixation can result in over-crosslinking and thus difficulty unmasking target nucleic acids, limiting access by probes to the target nucleic acids, while too brief fixation can lead to undesired nucleic acid degradation. Over-pretreatment can destroy cellular morphology, while under-pretreatment can result in poor access by probes to the target nucleic acids. Pretreatment regimens are often altered in an attempt to compensate for variability in samples and sample preparation techniques. The techniques described herein, however, permit a single pretreatment regimen to be employed for a wider variety of samples and sample preparation techniques while maintaining sensitivity and specificity of the assay.

The techniques described herein also facilitate use of more gentle pretreatment regimens than those currently employed for unmasking. For example, permeabilization of cells or tissues and unmasking of target nucleic acid for in situ detection is typically accomplished by heat treatment and incubation with a protease. A standard pretreatment for RNA ISH on an FFPE sample is to heat the sample in a citrate- or alkaline-based solution at 90-100° C. for 5-60 minutes, followed by treatment with proteinase K at a concentration of 1-12 μg/ml, e.g., at 37° for 10-40 minutes. Common pretreatment regimens for samples fixed by other methods (e.g., Bouin's, Zenker's, B-5, Clarke's, Carnoy's, or the like) and for fresh frozen samples also involve heat and/or protease treatment. Employing the techniques described herein (which, e.g., increase signal strength and tolerance for target masking) permits pretreatment to be performed under more gentle conditions, e.g., lower temperature and/or lower protease concentrations, thereby better preserving cell and tissue morphology.

An FFPE sample (e.g. after dewaxing and rehydration) may be prepared (pretreated) for hybridization with one or more label extenders without heat treatment and/or without protease treatment. An FFPE sample may be prepared for hybridization with one or more label extenders without a step in which the sample is exposed to a temperature over 90° C., over 85° C., over 80° C., over 75° C., over 70° C., over 60° C., over 55° C., over 50° C., over 45, over 40° C., over 37° C., over 35° C., over 30° C., over 25° C. or over 23° C. Preferably, a FFPE sample is prepared for hybridization with one or more label extenders without a step in which the sample is exposed to a temperature over 37° C. Additionally or alternatively, an FFPE sample may be prepared for hybridization with one or more label extenders without a step in which the sample is exposed to a protease or without a step in which the sample is exposed to a concentration of protease greater than 10 μg/ml, greater than 5 μg/ml, greater than 2.5 μg/ml, greater than 1 μg/ml, greater than 0.5 μg/ml, greater than 0.25 μg/ml or greater than 0.1 μg/ml. Preferably, if the sample is exposed to a protease, this step is performed for less than 60 minutes, less than 45 minutes or less than 30 minutes. Thus, in one class of embodiments, no heat treatment is applied to the sample during pretreatment, and unmasking is achieved through protease treatment. In another class of embodiments, pretreatment involves limited heat treatment (e.g., at 85° C. or less) followed by protease treatment. In another class of embodiments, pretreatment involves limited heat treatment (e.g., at 60-95° C., 50-85° C. or 55-85° C., preferably at 85° C. or less) with no exogenous protease being added to the sample. In one class of embodiments, pretreatment is omitted entirely. For example, addition and hybridization of label extenders and other oligonucleotide probes can immediately follow dewaxing, rehydration, and optionally drying of an FFPE sample or sectioning of a frozen sample. These types of pretreatment work well for a target whose expression level is high and for which many label extenders spanning a long targeted sequence can be designed.

Pretreatment of the sample may be performed by heating the sample in a citrate-based (acidic) solution or alkaline-based solution. As one specific example, pretreatment of the sample (after any dewaxing, rehydration, or drying or similar steps and prior to any hybridization steps) can be performed by heating the sample in a citrate- or alkaline-based solution at 50-85° C. (e.g., 55-85° C.) for 3-120 minutes (e.g., 5-60 minutes). This gentle heating step is optionally followed by protease treatment, e.g., with a lower concentration of protease such as proteinase K at less than 1 μg/ml at 37° C. for 10-40 minutes, e.g., 0.2-1 μg/ml, less than 500 ng/ml, 10-200 ng/ml, or 20-100 ng/ml. A gentler protease such as trypsin can be employed instead of proteinase K to better maintain morphology. As will be evident, the protease concentration and treatment time are balanced with the activity of the protease employed to achieve the desired results. Alternatively, no exogenous protease is added to the sample. This regimen can be employed, e.g., for dewaxed FFPE samples or samples fixed with other fixatives such as Bouin's, Zenker's, B-5, Clarke's, Carnoy's, or the like. Avoiding or limiting protease treatment is useful for simultaneous detection of protein and RNA in situ.

As another example, pretreatment can be performed by heating the sample at 55-100° C. for 5-60 minutes in an alkaline- or citrate-based solution. No exogenous protease is added to the sample. Again, this regimen can be employed, e.g., for dewaxed FFPE samples or samples fixed with other fixatives such as Bouin's, Zenker's, B-5, Clarke's, Carnoy's, or the like.

As yet another example, no heat is applied during pretreatment, and unmasking is achieved by protease treatment. After any dewaxing and rehydration or similar steps and prior to hybridization, the sample is incubated with one or more proteases, e.g., at a temperature of 37° C. or less as appropriate for the protease(s) employed (e.g., room temperature, 25° C. or less, or 23° C. or less). Suitable proteases are known in the art and include, but are not limited to, trypsin, pepsin, and protease type XIV from Streptomyces griseus. This regimen can be employed, e.g., for detection of microRNAs (miRNAs) or of RNA targets from samples in which the RNA has suffered degradation, e.g., dewaxed FFPE samples or other fixed samples. For detection from frozen samples embedded in OCT (optimal cutting temperature compound), proteinase K, e.g., at 20-100 ng/ml can be employed.

The sample may be pretreated by incubation in a solution comprising a detergent, surfactant or amphipathic glycoside. In another exemplary pretreatment regimen, the sample is incubated in a gentle permeabilization solution comprising a detergent or amphipathic glycoside at 0.01%-0.2% (v/v), e.g., 0.01%-0.2% (v/v) saponin, Triton™ X-100, digitonin, Leucoperm™, or Tween® 20, e.g., for 5-20 minutes at room temperature. Additional suitable detergents and amphipathic glycosides are known in the art. The solution optionally also includes 1-6% (v/v) formaldehyde and/or 50-85% (v/v) acetone or methanol. The solution can also include a buffer, salt, and the like, e.g., 1×PBS (phosphate-buffered saline). The permeabilization solution can be employed in the absence of any heat treatment, or can follow gentle heat treatment (e.g., at 50-85° C. for 3-120 minutes or 5-60 minutes). This regimen can be employed, e.g., for RNA ISH in combination with immunohistochemistry, since no exogenous protease is added. This regimen can also be employed for frozen samples, e.g., embedded in OCT, FFPE samples, or samples fixed with other fixatives.

It will be evident that pretreatment regimens limiting or eliminating heat treatment are readily applicable to RNA target nucleic acids. Double-stranded nucleic acid targets such as DNAs typically require denaturation of the target prior to hybridization with the label extenders and label probe system, e.g., by chemical denaturation or heating (e.g., for 5-10 minutes at 95-100° C. or 2-4 minutes at 90-93° C.).

### Compositions

Compositions related to the methods are another feature of the invention. Thus, one general class of embodiments provides a composition for detecting a target nucleic acid. The composition includes one or more label extenders and a preamplifier. Each label extender is configured to hybridize to the target nucleic acid and to two or more copies of the preamplifier. The preamplifier is configured to hybridize to a single one of the label extenders. The composition optionally also includes at least one intermediate amplifier, an amplification multimer, and/or a label probe. The composition optionally includes the target nucleic acid. For example, the composition can include a sample that comprises a cell comprising the target nucleic acid. As noted above, the label probe comprises or is configured to bind to a label. In one class of embodiments, the label is an enzyme, and the composition optionally includes a chromogenic substrate for the enzyme.

Essentially all of the features noted for the methods above apply to these compositions as well, as relevant; for example, with respect to configuration of the label extenders, composition of the label probe system, type of label, source of the sample and/or nucleic acids, and/or the like.

A related general class of embodiments provides a composition comprising one or more label extenders and a preamplifier. Each of the label extenders comprises a polynucleotide sequence L-1 that is complementary to a polynucleotide sequence in the target nucleic acid, wherein L-1 is less than 15 nucleotides in length. Each label extender is also configured to hybridize to one or more copies of the preamplifier (e.g., to two or more copies of the preamplifier). The preamplifier is configured to hybridize to a single one of the label extenders. The composition optionally also includes at least one intermediate amplifier, an amplification multimer, and/or a label probe. The composition optionally includes the target nucleic acid. For example, the composition can include a sample that comprises a cell comprising the target nucleic acid. As noted above, the label probe comprises or is configured to bind to a label. In one class of embodiments, the label is an enzyme, and the composition optionally includes a chromogenic substrate for the enzyme.

Essentially all of the features noted for the methods above apply to these embodiments as well, as relevant; for example, with respect to configuration of the label extenders, composition of the label probe system, type of label, source of the sample and/or nucleic acids, and/or the like.

### Kits

Yet another general class of embodiments provides a kit for detecting a target nucleic acid. The kit includes one or more label extenders, a preamplifier, an amplification multimer, and a label probe, packaged in one or more containers. Each label extender is configured to hybridize to the target nucleic acid and to two or more copies of the preamplifier. The preamplifier is configured to hybridize to a single one of the label extenders. The kit optionally also includes at least one intermediate amplifier.

As noted above, the label probe comprises or is configured to bind to a label. In one class of embodiments, the label is an enzyme, and the kit optionally includes a chromogenic substrate for the enzyme.

The kit optionally also includes instructions for using the kit to detect the target nucleic acid (e.g., in situ), one or more buffered solutions (e.g., diluent, hybridization buffer, and/or wash buffer), pretreatment reagents, standards comprising one or more nucleic acids at known concentration, and/or the like.

Essentially all of the features noted for the methods above apply to these embodiments as well, as relevant; for example, with respect to configuration of the label extenders, composition of the label probe system, type of label, source of the target nucleic acid, and/or the like.

A related general class of embodiments provides a kit for detecting a target nucleic acid. The kit includes one or more label extenders, a preamplifier, an amplification multimer, and a label probe. Each of the label extenders comprises a polynucleotide sequence L-1 that is complementary to a polynucleotide sequence in the target nucleic acid, wherein L-1 is less than 15 nucleotides in length. Each label extender is also configured to hybridize to one or more copies of the preamplifier (e.g., to two or more copies of the preamplifier). The preamplifier is configured to hybridize to a single one of the label extenders. The kit optionally also includes at least one intermediate amplifier.

As noted above, the label probe comprises or is configured to bind to a label. In one class of embodiments, the label is an enzyme, and the kit optionally includes a chromogenic substrate for the enzyme.

The kit optionally also includes instructions for using the kit to detect the target nucleic acid (e.g., in situ), one or more buffered solutions (e.g., diluent, hybridization buffer, and/or wash buffer), pretreatment reagents, standards comprising one or more nucleic acids at known concentration, and/or the like.

Essentially all of the features noted for the methods above apply to these embodiments as well, as relevant; for example, with respect to configuration of the label extenders, composition of the label probe system, type of label, source of the target nucleic acid, and/or the like.

### Systems

In one aspect, the invention includes systems, e.g., systems used to practice the methods herein and/or comprising the compositions described herein. The system can include, e.g., a fluid handling element, a fluid and/or slide containing element, a heating element, a temperature controller, a detector (e.g., a bright-field microscope), and/or a robotic element that moves other components of the system from place to place as needed (e.g., a slide handling element). For example, in one class of embodiments, a composition of the invention is contained in a BOND-III or BOND RX or like instrument for automated in situ hybridization staining and processing.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, a heating element, and/or robotic element). The computer can also receive data from other components of the system, e.g., from a detector, and can interpret the data, provide it to a user in a human readable format, or use that data to initiate further operations, in accordance with any programming by the user.

### Labels

A wide variety of labels are well known in the art and can be adapted to the practice of the present invention. For example, conjugation of enzymes such as alkaline phosphatase and horseradish peroxidase to polynucleotide probes has been described, as have appropriate substrates such as fast red and fast blue (for alkaline phosphatase) and 3,3′-diaminobenzidine (DAB, for horseradish peroxidase) for detection of such enzymes. See, e.g., U.S. Pat. Nos. 5,780,227 and 7,033,758.

As another example, a number of fluorescent labels are well known in the art, including but not limited to, hydrophobic fluorophores (e.g., phycoerythrin, rhodamine, Alexa Fluor 488 and fluorescein), green fluorescent protein (GFP) and variants thereof (e.g., cyan fluorescent protein and yellow fluorescent protein), and quantum dots. See e.g., The Handbook: A Guide to Fluorescent Probes and Labeling Technologies, Eleventh Edition or Web Edition (2014) from Invitrogen (available on the world wide web at www (dot) lifetechnologies (dot) com/us/en/home/references/molecular-probes-the-handbook) for descriptions of fluorophores emitting at various different wavelengths (including tandem conjugates of fluorophores that can facilitate simultaneous excitation and detection of multiple labeled species). For use of quantum dots as labels for biomolecules, see e.g., Dubertret et al. (2002) Science 298:1759; Nature Biotechnology (2003) 21:41-46; and Nature Biotechnology (2003) 21:47-51. As yet another example, luminescent labels and light-scattering labels (e.g., colloidal gold particles) have been described. See, e.g., Csaki et al. (2002) “Gold nanoparticles as novel label for DNA diagnostics” Expert Rev Mol Diagn 2:187-93.

Labels can be introduced to molecules, e.g. polynucleotides, during synthesis or by postsynthetic reactions by techniques established in the art; for example, kits for fluorescently labeling polynucleotides with various fluorophores are available from Molecular Probes, Inc. (www (dot) molecularprobes (dot) com), and fluorophore-containing phosphoramidites for use in nucleic acid synthesis are commercially available. Techniques for covalently coupling an enzyme to an oligonucleotide are well known in the art. For example, a homobifunctional or heterobifunctional cross linker can be employed to covalently couple an enzyme with an amine-, thiol-, or aldehyde-modified oligonucleotide. Similarly, techniques for noncovalently coupling an enzyme to an oligonucleotide are also well known in the art. For example, streptavidin-conjugated enzymes (e.g., streptavidin-conjugated alkaline phosphatase or horseradish peroxidase) are commercially available and can be bound to a biotinylated oligonucleotide probe.

Similarly, signals from the labels (e.g., a colored product from a chromogenic substrate, light emission from a luminescent product, absorption by and/or fluorescent emission from a fluorescent label) can be detected by essentially any method known in the art. For example, microscopy (e.g., bright-field microscopy), multicolor detection, detection of FRET, fluorescence polarization, and the like are well known in the art.

### Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N. Y., 2000 and Current Protocols in Molecular Biology, F. M. Ausubel et al., eds., Current Protocols, a joint venture between Greene Publishing Associates, Inc. and John Wiley & Sons, Inc., (supplemented through 2014). Other useful references, e.g. for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla.

### Probe Design

Design of label extenders for use in the present invention has been detailed above. As noted, for a given target nucleic acid, the corresponding label extenders, optional capture extenders, and optional blocking probes are preferably complementary to physically distinct, nonoverlapping sequences in the nucleic acid, which can but need not be contiguous. The Tₘs of the label extender-nucleic acid, capture extender-nucleic acid, and blocking probe-nucleic acid complexes are preferably greater than the hybridization temperature, e.g., by 5° C. or 10° C. or preferably by 15° C. or more, such that these complexes are stable at the hybridization temperature. For example, the Tₘs of the label extender-target nucleic acid complexes can be between 58-72° C. or between 62-67° C. where the hybridization temperature for in situ detection of the target is 40° C. Potential label extender and optional capture extender sequences (e.g., potential sequences L-1 and C-3) are optionally examined for possible interactions with label extenders, capture extenders, label probe system components (e.g., preamplifier, intermediate amplifier, amplification multimer, and/or label probe), other target nucleic acids in a multiplex assay format, and/or any relevant genomic sequences, for example. Other probes can be similarly examined (e.g., the preamplifier, optional capture probes, and the like). Sequences expected to cross-hybridize with undesired nucleic acids are typically not selected for use in the label extenders or other probes. See, e.g., Player et al. (2001) “Single-copy gene detection using branched DNA (bDNA) in situ hybridization” J Histochem Cytochem 49:603-611. Examination can be, e.g., visual (e.g., visual examination for complementarity), computational (e.g., computation and comparison of binding free energies), and/or experimental (e.g., cross-hybridization experiments).

Also as noted, a label extender optionally comprises at least one non-natural nucleotide. Non-natural nucleotides can similarly be included in the preamplifiers, intermediate amplifiers, amplification multimers, label probes, capture extenders, and/or capture probes, if desired. Use of such non-natural base pairs (e.g., isoG-isoC base pairs) in probes that hybridize to each other (e.g., label extenders and preamplifiers) can, for example, reduce background and/or simplify probe design by decreasing cross hybridization, or it can permit use of shorter label extenders or other probes when the non-natural base pairs have higher binding affinities than do natural base pairs (including the bases typical to biological DNA or RNA, i.e., A, C, G, T, or U). Examples of non-natural nucleotides include, but are not limited to, constrained ethyl analogs (see, e.g., U.S. Pat. No. 7,572,582, U.S. Pat. No. 6,670,461, U.S. Pat. No. 6,794,499, U.S. Pat. No. 7,034,133, U.S. Pat. No. 5,700,637, U.S. Pat. No. 5,436,327, and U.S. Pat. No. 7,399,846), locked nucleic acid nucleotides (available from Exiqon A/S, www (dot) exiqon (dot) com; see, e.g., SantaLucia Jr. (1998) Proc Natl Acad Sci 95:1460-1465), and isoG, isoC, and other nucleotides used in the AEGIS system (Artificially Expanded Genetic Information System, available from EraGen Biosciences, www (dot) eragen (dot) com; see, e.g., U.S. Pat. No. 6,001,983, U.S. Pat. No. 6,037,120, and U.S. Pat. No. 6,140,496).

### Making Polynucleotides

Methods of making nucleic acids (e.g., by in vitro amplification, purification from cells, or chemical synthesis), methods for manipulating nucleic acids (e.g., by restriction enzyme digestion, ligation, etc.) and various vectors, cell lines and the like useful in manipulating and making nucleic acids are described in the above references. In addition, methods of making branched polynucleotides (e.g., amplification multimers) are described in U.S. Pat. No. 5,635,352, U.S. Pat. No. 5,124,246, U.S. Pat. No. 5,710,264, and U.S. Pat. No. 5,849,481, as well as in other references mentioned above.

In addition, essentially any polynucleotide (including, e.g., labeled or biotinylated polynucleotides) can be custom or standard ordered from any of a variety of commercial sources, such as The Midland Certified Reagent Company (www (dot) mcrc (dot) com), The Great American Gene Company (www (dot) genco (dot) com), ExpressGen Inc. (www (dot) expressgen (dot) com), Qiagen (oligos (dot) qiagen (dot) com) and many others.

A label, biotin, or other moiety can optionally be introduced to a polynucleotide, either during or after synthesis. For example, a biotin phosphoramidite can be incorporated during chemical synthesis of a polynucleotide. Alternatively, any nucleic acid can be biotinylated using techniques known in the art; suitable reagents are commercially available, e.g., from Pierce Biotechnology (www (dot) piercenet (dot) com). Similarly, any nucleic acid can be fluorescently labeled, for example, by using commercially available kits such as those from Molecular Probes, Inc. (www (dot) molecularprobes (dot) com) or Pierce Biotechnology (www (dot) piercenet (dot) com) or by incorporating a fluorescently labeled phosphoramidite during chemical synthesis of a polynucleotide.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Examples 1

### In Situ Hybridizations

The following sets forth a series of experiments that demonstrate in situ detection of nucleic acids. Detection using different label extender configurations is compared, for targets of different sizes and expression levels.

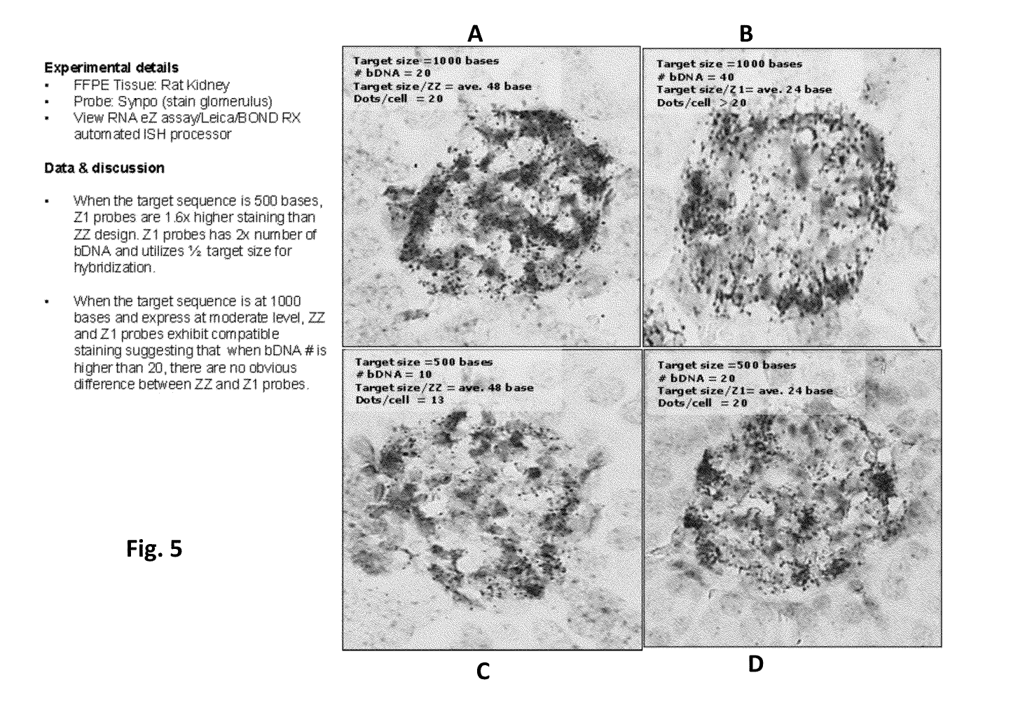

FIG. 5 compares detection of relatively large targets using two different label extender configurations, ZZ (where two label extenders are required to capture a copy of the preamplifier to the target, Panels A and C) and Z1 (where each label extender hybridizes to a single preamplifier and each preamplifier hybridizes to a single label extender, Panels B and D). The target nucleic acid in this example is rat Synpo. In Panels A and B, the label extenders hybridize within a 1000 base region of the target. In Panels C and D, the label extenders hybridize within a shorter, 500 base region of the target (illustrating detection of a shorter target), and the Z1 label extender configuration, which permits capture of at least twice as many copies of the label probe system, exhibits higher staining.

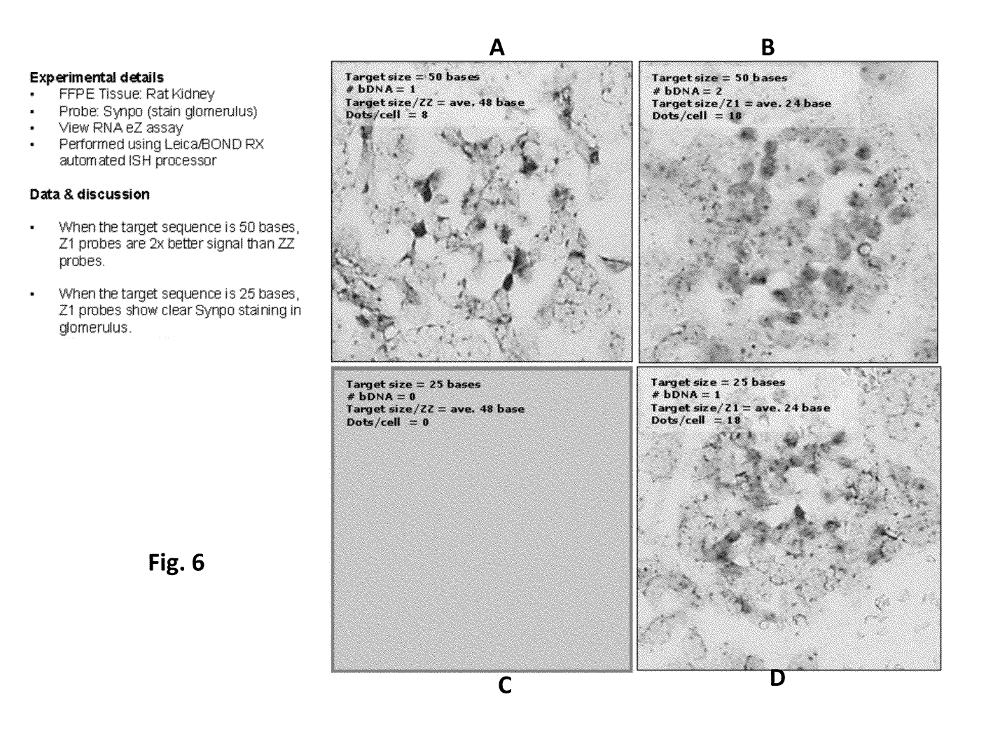

FIG. 6 compares detection of small targets using two different label extender configurations, ZZ (Panels A and C) and Z1 (Panels B and D). In Panels A and B, the label extenders hybridize within a 50 base region of the target, rat Synpo. The Z1 label extender configuration exhibits higher signal. In Panels C and D, the label extenders hybridize within a 25 base region of the target. The ZZ label extenders are unable to detect the 25 base region (thus, panel 6C is not provided with an image), since each label extender hybridizes to approximately 24 bases of the target and binding of two label extenders is required for capture of the label probe system.

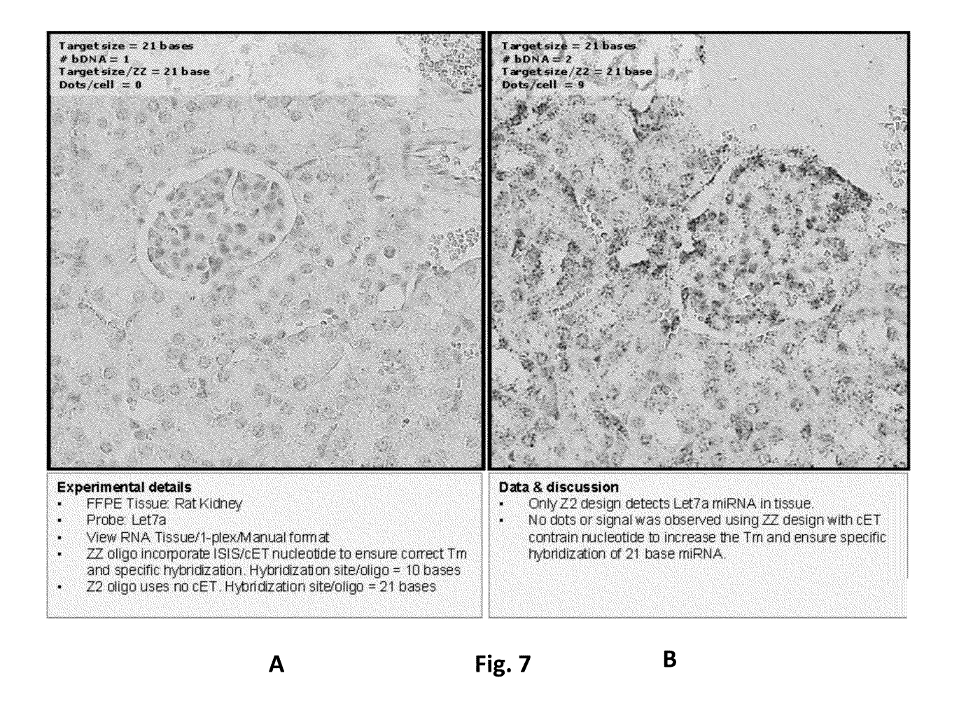

FIG. 7 compares in situ detection of Let7a microRNA (miRNA) using two different label extender configurations, ZZ (Panel A) and Z2 (where each label extender hybridizes to two copies of the preamplifier and each copy of the preamplifier hybridizes to a single label extender, Panel B). Even though the two ZZ label extenders include a 10 base L-1 sequence including constrained ethyl nucleotides to achieve stable hybridization with the target miRNA and together should capture a single copy of the preamplifier to the target, no signal is observed. The single Z2 label extender, which includes a 21 base L-1 sequence and which should capture two copies of the preamplifier to the target, does permit detection of the miRNA.

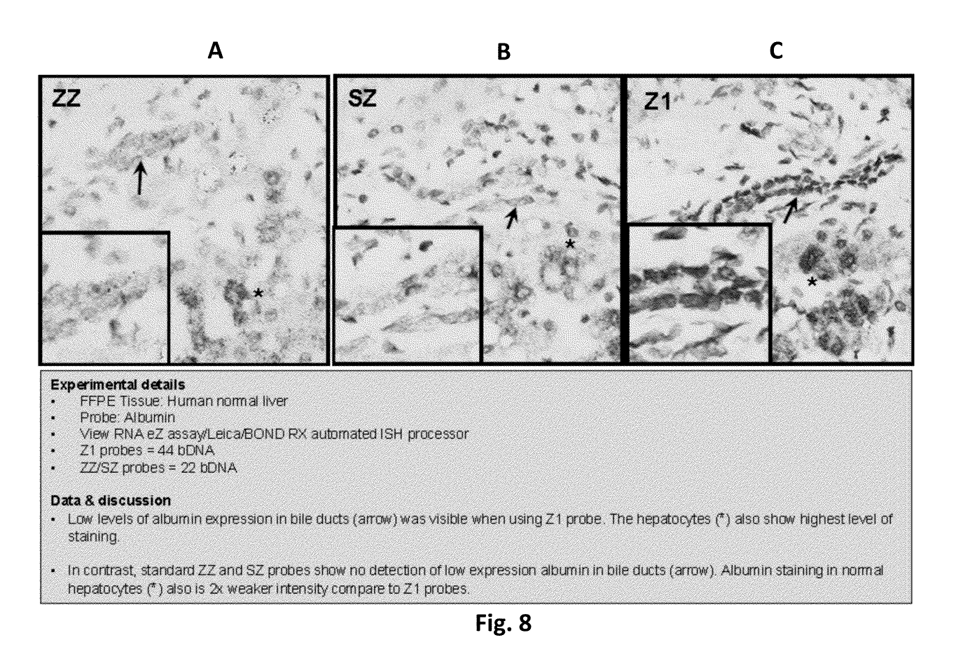

FIG. 8 compares detection of low levels of albumin in situ in bile duct using three different label extender configurations, ZZ (Panel A), SZ (a different configuration in which two label extenders are also required to capture a copy of the preamplifier to the target, Panel B), and Z1 (Panel C). The Z1 label extender configuration detects albumin expression in bile ducts as well as in hepatocytes, while the ZZ and SZ configurations, which capture at best half as much label to the target, only detect expression in hepatocytes.

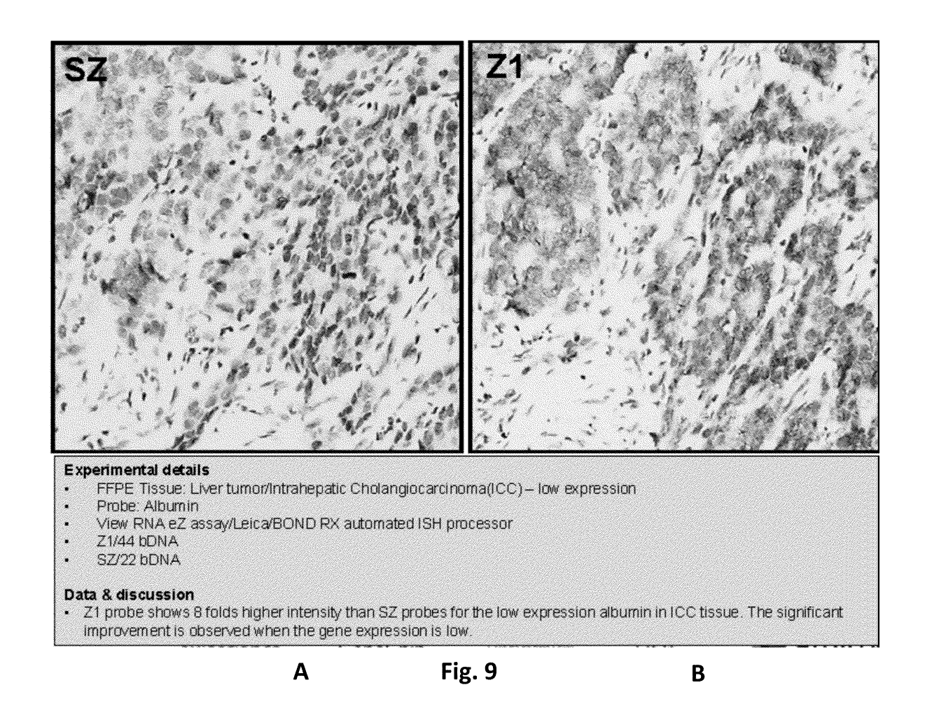

FIG. 9 compares detection of low levels of albumin in situ using two different label extender configurations, SZ (Panel A) and Z1 (Panel B). The Z1 configuration detects albumin expression with a signal eight fold higher than does the SZ configuration, despite theoretically only capturing at most twice as much label to the target mRNA (with respect to the number of preamplifiers binding to the target region under an assumption that the entire target region will be sufficiently unmasked to allow hybridization of the label extenders).

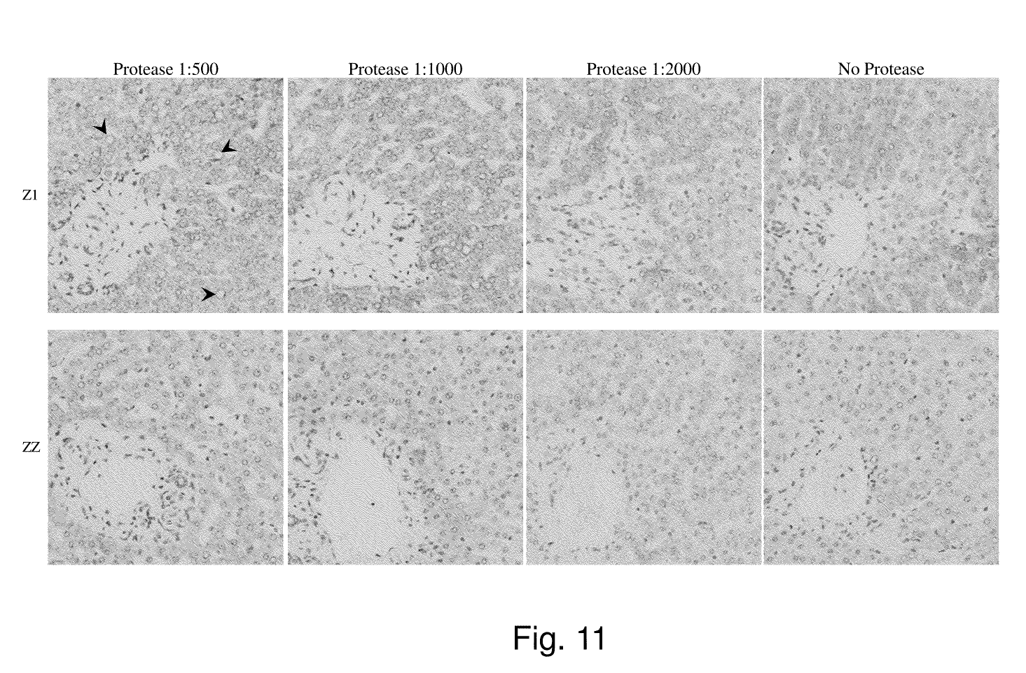

FIG. 11 presents in situ hybridization of albumin mRNA transcript target nucleic acid in liver tissue with detection using either double label extender bDNA systems or single label extender systems. Pretreatment used Leica Biosystems Bond™ Epitope Retrieval Solution 1 at pH 5.9-6.1 at 25° C. with citrate based buffer and surfactant, and the Leica Biosystems Bond™ Enzyme Pretreatment Kit with protease at a 17 mg/ml stock solution and enzyme diluents containing Tris-buffered saline, surfactant and 0.35% ProClin™ 950. The 1:500, 1:1000 and 1:2000 protease dilutions were made from the aforementioned stock solution. Protease digestion time was 30 min for all dilutions. Note the relatively gentle pretreatment conditions result in a relatively weak signal when probing with the system based on a double label extender format (lower micrographs), but a robust signal entirely adequate for clinical interpretation was obtained under the same conditions where the assay uses a single label extender format (upper micrographs). Also note that the most gentle protease treatment (no protease) in combination with single label extender detection provided a stronger signal than the most stringent protease treatment in combination with the double label extender detection; while the no protease also provided the benefit of better tissue and cell morphology preservation. The photomicrographs are based on human liver processed using ER1 epitope retrieval buffer and ViewRNA eZ-L Detection kit on the Leica Bond III automated ISH staining instrument. Images taken at 20× objective magnification.

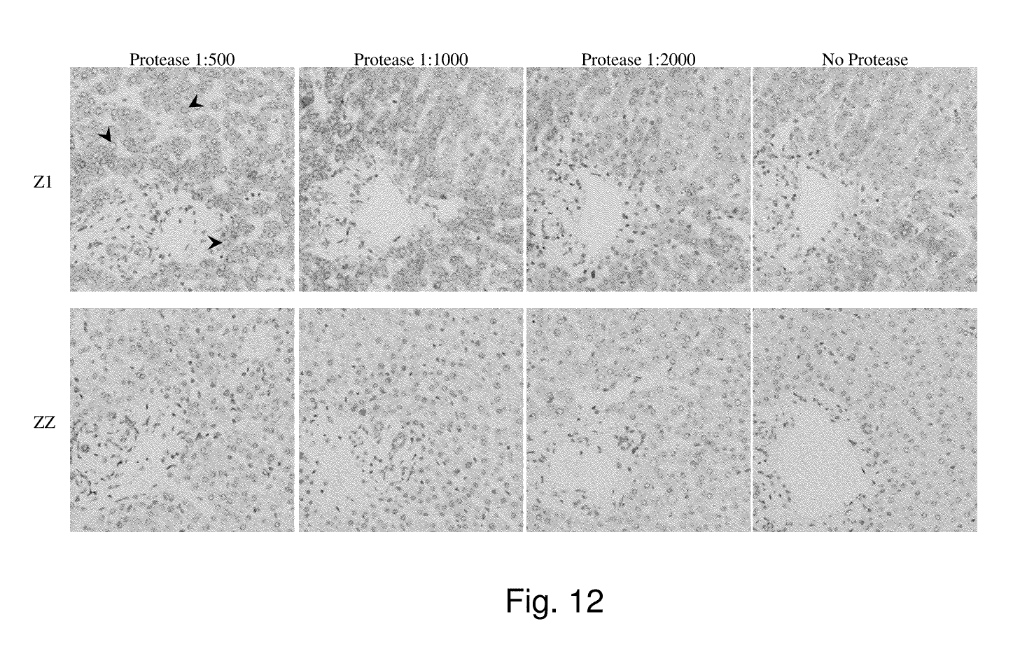

FIG. 12 presents in situ hybridization of a albumin mRNA transcript target nucleic acid in liver tissue with detection using either double label extender bDNA systems or single label extender systems using Leica Biosystems Bond™ Epitope Retrieval Solution 2 pretreatment at pH 8.9-9.1 at 25° C. with EDTA based buffer and surfactant, and the Leica Biosystems Bond™ Enzyme Pretreatment Kit with protease at a 17 mg/ml stock solution and enzyme diluents containing Tris-buffered saline, surfactant and 0.35% ProClin™ 950. The 1:500, 1:1000 and 1:2000 protease dilutions were made from the aforementioned stock solution. Protease digestion time was 30 min for all dilutions. Again, the more gentle pretreatment conditions result in a relatively weak signal when probing with the system based on a double label extender format (lower micrographs), while the single label extender system (upper micrographs) provided adequate robust signal entirely adequate for clinical interpretation with the tissue retaining more natural morphology.

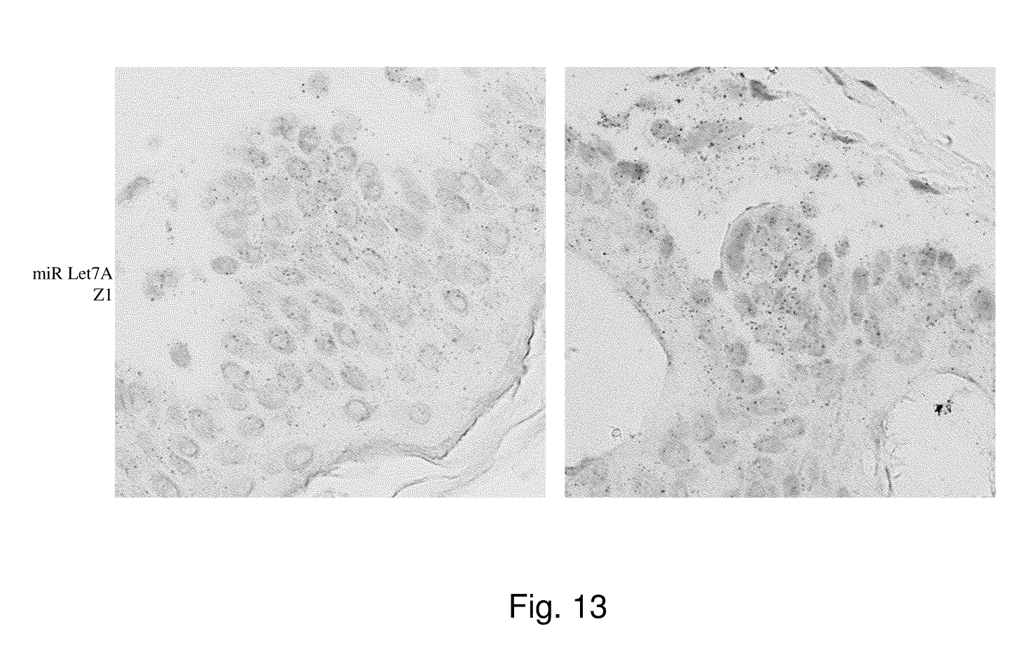

FIG. 13 presents in situ hybridization of a micro-RNA target in human epidermis using a single label extender bDNA system. The Let7a miRNA target nucleic acid strand is 22 bases in length. The challenging target would not be detectable in a double label extender system. Here, the system is able to present robust signal associated with the moderate expression of Let7a with 6-20 dots/cell using a single label extender system with a single Z1 label extender. Here human skin is processed with 1:150 dilution of protease (from a stock concentration of 2.7 mg/ml) using the ViewRNA Manual Detection kit. Images taken at 40× objective magnification.

### Examples 2

### Gentle Pretreatments

Pretreatment, before in situ bDNA hybridization steps, is intended to open the cell/tissue matrix to the nucleic acid probes, without damaging tissue morphology to the point that a particular microscopic analysis becomes difficult to interpret. A trade off often exists wherein a more stringent pretreatment opens the sample to more probe hybridization and a stronger hybridization signal, while damaging the microscopic appearance of the sample. Where the pathologist or scientist is mainly concerned with determining a quantity or presence of a target nucleic acid, the location of the signal relative to cell structures may be less important. On the other hand, where the combination of signal and location is important, one may want to preserve more of the cell/tissue structures through the stresses of pretreatment. For example whereas a particular target nucleic acid may be of no concern in a normal blast cell, it may be of more interest in a possibly malignantly transformed cell.

In order to attain a desired level of pretreatment, a number of parameters can be adjusted. For example, the amount of morphology loss can be influenced by the pretreatment pH, temperatures, intensity (including enzyme concentration) and time of protease and other enzymatic and non-enzymatic permeabilization treatments, exposure to organic solvents, extent of cross-linking, and time frame of pretreatment exposures. The damage to morphology can also depend on the specific cells or tissues to be assayed.

We have reviewed several tissues and identified conditions that typically result in strong bDNA signals on in situ detections of tissues on microscope (clinical) slides. From these starting points, one can confirm a specific signal is be generated under initial pretreatment conditions. If the pretreatment has excessively damaged microscopic morphology of the sample, one can elect a more gentle pretreatment condition. Typically, one can reduce the time exposed to protease (as well as further dilution of the protease) and/or reduce the temperature of heat pretreatments to improve morphology retention while retaining adequate signal. In many situations, times of treatment can be extended while reducing temperature and/or protease even more, for improved morphology without significant loss of signal. For example, permeability of the sample to probes may be retained while tripling pretreatment time, lowering temperature 10° C. and cutting protease levels in half.

We have found that certain tissues can be assayed, e.g., using bDNA techniques with a minimum of pretreatments. These include soft and homogenous tissues such as lymph node, pancreas, placenta, spleen, tonsil, and xenograft. Slides of such tissues can be pretreated with relatively gentle procedures such as, e.g., slightly alkaline pH, temperatures below 90° C. for 10 minutes or less, and proteinase K diluted 1:1000 or more (from a stock solution of 17 mg/ml).

More solid and heterogeneous tissues can be assayed, e.g., using bDNA techniques with a slightly less gentle of pretreatments. These tougher tissues include brain, breast, colon, embryo, kidney, lung, skin, spinal cord, stomach, testis, and thyroid. Slides of such tissues can be pretreated with slightly less gentle procedures such as, e.g., slightly acidic, heat induced epitope retrieval at temperatures below 95° C. for 10 minutes or less, and proteinase K diluted 1:1000 or more (such as 1:1,500, 1:2,000, 1:2,500 and 1:3,000) from a stock solution of, e.g., 17 mg/ml.

Certain tissues may require increased pretreatment times. For example, cervix/uterus, eye/retina, gall bladder, heart, muscles (smooth/skeletal), ovary, prostate, and urinary bladder slides can be pretreated with slightly less gentle procedures such as, e.g., slightly acidic, heat induced epitope retrieval at temperatures below 95° C. for 10 minutes or less, but proteinase K at a higher concentration or for a longer digestion period. Finally, we have found that liver tissue may require even more stringent pretreatment (e.g., 30 minutes in protease) to obtain a strong detection signal.

For these tissues in these conditions, on confirmation of adequate specific assay detection signal, we suggest the following further gentle pretreatments, should one desire better preservation of cell and/or tissue morphology: a 10° C. temperature reduction for the heat induced epitope retrieval step, 50% reduction of protease concentration, elimination of organic solvent treatments, and/or a 50% reduction in cross linking time or reagent concentration.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of detecting a target nucleic acid in situ in a cell or tissue, the method comprising:
providing a cell or tissue sample comprising the target nucleic acid;
hybridizing one or more label extenders to one or more target regions of the target nucleic acid;
hybridizing one or more copies of a preamplifier to each of the label extenders, wherein each copy of the preamplifier hybridizes to a single one of the label extenders;
binding multiple copies of a label to each copy of the preamplifier; and
detecting a signal produced by the labels, thereby detecting the target nucleic acid.

2. The method of claim 1, comprising hybridizing two or more copies of the preamplifier to each of the label extenders.

3. The method of claim 1, wherein each of the label extenders comprises a polynucleotide sequence L-1 that is complementary to a polynucleotide sequence in the target nucleic acid, wherein L-1 is less than 15 nucleotides in length.

4. The method of claim 1, comprising hybridizing two or more different label extenders to noncontiguous regions of the target nucleic acid.

5. The method of claim 1, wherein the target nucleic acid is a micro RNA (miRNA).

6. The method of claim 1, wherein the target region is less than 400 nucleotides in length.

7. The method of claim 1, wherein the target region is 300 nucleotides or less in length.

8. The method of claim 1, wherein the target region is 25 nucleotides or less in length.

9. The method of claim 1, wherein the sample comprises a cell comprising the target nucleic acid; and the method comprises hybridizing the one or more label extenders to the target nucleic acid in the cell.

10. The method of claim 9, wherein the sample comprises a tissue section comprising the cell.

11. The method of claim 10, wherein the tissue section is a formalin-fixed, paraffin-embedded tissue section.

12. The method of claim 9, wherein the sample is maintained at a temperature of 85° C. or less prior to the hybridizing, binding, and detecting steps.

13. The method of claim 12, wherein the sample is maintained at a temperature between 50° C. and 85° C. for 3 to 120 minutes prior to the hybridizing, binding, and detecting steps.

14. The method of claim 12, wherein the sample is a formalin-fixed, paraffin-embedded tissue section, the method comprising dewaxing and rehydrating the sample prior to the hybridizing, binding, and detecting steps; wherein following the dewaxing and rehydrating step and prior to the hybridizing, binding, and detecting steps, the sample is maintained at a temperature of 37° C. or less.

15. The method of claim 12, wherein the sample is incubated with proteinase K at a concentration of less than 1 μg/ml prior to the hybridizing, binding, and detecting steps.

16. The method of claim 12, wherein the sample is incubated with proteinase K at a concentration of 20-100 ng/ml prior to the hybridizing, binding, and detecting steps.

17. The method of claim 12, wherein no exogenous protease is added to the sample.

18. The method of claim 17, wherein the sample is incubated in a solution comprising a detergent or amphipathic glycoside at 0.01%-0.2% (v/v) prior to the hybridizing, binding, and detecting steps.

19. The method of claim 9, comprising washing the cell without agitation to remove materials that are not hybridized or bound to the target nucleic acid.

20. The method of claim 1, wherein the label is an enzyme.

21. The method of claim 20, wherein detecting a signal produced by the label comprises providing a chromogenic substrate for the enzyme and detecting a colored product produced by action of the enzyme on the substrate.

22. The method of claim 1, wherein binding multiple copies of the label to each copy of the preamplifier comprises:
hybridizing multiple copies of an intermediate amplifier to each copy of the preamplifier;
hybridizing multiple copies of an amplification multimer to each copy of the intermediate amplifier; and
hybridizing multiple copies of a label probe to each copy of the amplification multimer, wherein each copy of the label probe comprises a copy of the label.

23. The method of claim 1, wherein binding multiple copies of the label to each copy of the preamplifier comprises:
hybridizing multiple copies of an intermediate amplifier to each copy of the preamplifier;
hybridizing multiple copies of an amplification multimer to each copy of the intermediate amplifier;
hybridizing multiple copies of a label probe to each copy of the amplification multimer; and
binding a copy of the label to each copy of the label probe.

24. The method of claim 1, wherein the target nucleic acid is degraded or masked.

25. The method of claim 1, further comprising a gentle pretreatment of the cells or tissues; wherein the pretreatment comprises exposing the cells or tissues to less than 1 μg/ml of protease at a temperature of 85° C. or less for 10 minutes or less.

26. The method of claim 1, further comprising a gentle pretreatment of the cells or tissues; wherein the pretreatment comprises other than exposure of the cells or tissues to an organic solvent or exposure to an aldehyde.

27. A method of detecting a target nucleic acid in situ, the method comprising:
providing a sample that comprises a cell comprising the target nucleic acid;
hybridizing one or more label extenders to the target nucleic acid in the cell, wherein each of the label extenders comprises a polynucleotide sequence L-1 that is complementary to a polynucleotide sequence in the target nucleic acid, wherein L-1 is less than 15 nucleotides in length;
hybridizing one or more copies of a preamplifier to each of the label extenders, wherein each copy of the preamplifier hybridizes to a single one of the label extenders;
binding multiple copies of a label to each copy of the preamplifier; and
detecting a signal produced by the label, thereby detecting the target nucleic acid.

28. The method of claim 27, comprising hybridizing two or more copies of the preamplifier to each of the label extenders.

29. The method of claim 27, wherein the label is an enzyme, and wherein detecting a signal produced by the label comprises providing a chromogenic substrate for the enzyme and detecting a colored product produced by action of the enzyme on the substrate.

30. A method of detecting a target nucleic acid in situ, the method comprising:
providing a formalin-fixed, paraffin-embedded sample that comprises a cell comprising the target nucleic acid;
dewaxing and rehydrating the sample to provide a dewaxed sample that comprises the cell comprising the target nucleic acid;
hybridizing one or more label extenders to the target nucleic acid in the cell in the dewaxed sample;
hybridizing one or more copies of a preamplifier to each of the label extenders, wherein each copy of the preamplifier hybridizes to a single one of the label extenders without hybridizing to a another label extender;
binding multiple copies of a label to each copy of the preamplifier; and
detecting a signal produced by the label, thereby detecting the target nucleic acid;
wherein the dewaxed sample is maintained at a temperature of 85° C. or less prior to the hybridizing, binding, and detecting steps.

31. The method of claim 31, wherein each of the label extenders comprises a polynucleotide sequence L-1 that is complementary to a polynucleotide sequence in the target nucleic acid, wherein L-1 is less than 15 nucleotides in length.

32. The method of claim 32, further comprising a pretreatment comprises exposing the cell to less than 1 μg/ml of protease for 10 minutes or less.
